(19) United States
(12) Patent Application Publication
(10) Pub. No.: US 2009/0208539 A1
(43) Pub. Date: Aug. 20, 2009

(54) STABLE ATORVASTATIN FORMULATIONS

(76) Inventors: Adel Penhasi, Holon (IL); Yaakov Stephane Attali, Jerusalem (IL)

Correspondence Address: WINSTON & STRAWN LLP;PATENT DEPARTMENT, 1700 K STREET, N.W., WASHINGTON, DC, 20006, US

(21) Appl. No.: 11/719,791

(22) PCT Filed: Nov. 22, 2005
(86) PCT No.: PCT/IL05/01235

§ 371 Date: Mar. 26, 2009

Related U.S. Application Data
(60) Provisional application No. 60/629,412, filed on Nov. 22, 2004.

(51) Int. Cl. A61K 9/00; A61K 31/40

(52) U.S. Cl. 424/400; 514/423

(57) ABSTRACT

A simple yet efficient formulation for providing excellent bioefficacy, wherein the formulation includes atorvastatin or a salt thereof, in crystalline or amorphous form, with at least one pharmaceutical excipient selected to a form of atorvastatin that has beneficial properties, such as enhanced stability. These formulations do not need to include a stabilizer.

### FIELD OF THE INVENTION

The present invention relates to a simple and elegant stable pharmaceutical formulation for atorvastatin and optionally its pharmaceutically acceptable salts thereof.

### BACKGROUND

Atorvastatin-[R-R*,R*)]-2-(4-Fluorophenyl)-β,δ-dihydroxy-5-(1-methylethyl)-3-phenyl-4-[(phenylamino)-carbonyl]-1H-pyrrole-1-heptanoic acid and pharmaceutically acceptable salts thereof (see for example U.S. Pat. No. 5,273,995 to Warner-Lambert, hereby incorporated by reference as if fully set forth herein) is a well-known lipid lowering agent.

Atorvastatin is an inhibitor of 3 hydroxy-3-methylglutaryl-coenzyme A (HMG-CoA) reductase. This enzyme catalyzes the conversion of HMG-CoA to mevalonate, an early and rate-limiting step in cholesterol biosynthesis. It is usually given orally.

One optional but preferred form of atorvastatin is the pharmaceutically acceptable hemi-calcium salt form, atorvastatin calcium, because it has good stability and bioefficacy. Atorvastatin calcium is a white to off-white powder that is nearly insoluble in aqueous solutions of pH 4 and below, which are the conditions typically present in the stomach of a subject. Atorvastatin calcium is very slightly soluble in distilled water, pH 7.4 phosphate buffer, and acetonitrile, slightly soluble in ethanol, and freely soluble in methanol. If incorporated in a fast release solid dosage form (similar to Lipitor®), atorvastatin calcium should be used as a micronized powder to enhance its speed of dissolution because of the poor solubility properties of this material in aqueous systems such as those existing in the GI tract. Such micronized material is not suitable for dry mix process and should preferably be wet granulated and dried with part of the other excipients of the formula to avoid aggregation of the hydrophobic Atorvastatin calcium particles on dissolution and ensure a fast dissolution profile.

Different crystal and amorphous forms of atorvastatin have been described. Atorvastatin lactone was first disclosed to the public and claimed in U.S. Pat. No. 4,681,893. The hemi calcium salt—atorvastatin calcium—is disclosed in U.S. Pat. No. 5,273,995. This patent teaches that the calcium salt is obtained by crystallization from a brine solution resulting from the transposition of the sodium salt with CaC12 and further purified by recrystallization from a 5:3 mixture of ethyl acetate and hexane. Both of these U.S. Patents are hereby incorporated by reference.

U.S. Pat. Nos. 5,003,080; 5,097,045; 5,103,024; 5,124,482; 5,149,837; 5,155,251; 5,216,174; 5,248,,793; 5,280,132; 5,342,952; 5,007,080; 6,274,740; which are herein incorporated by reference, describe various processes and key intermediates for preparing atorvastatin calcium. All these processes give mixture of crystalline and amorphous forms.

U.S. Pat. No. 5,969,156 discloses three polymorphs of atorvastatin calcium designated Forms I, II, and IV by the inventors of those forms. Though the inventors claim certain processing and therapeutic advantages of their forms over the amorphous atorvastatin calcium, advantages may yet be realized by other heretofore undiscovered forms of atorvastatin calcium.

PCT application WO 97/03960 and PCT application WO 00/71116 describes method for the production of amorphous atorvastatin calcium.

PCT application W097/03958 and U.S. Pat. No. 6,121,461 disclose the method for the preparation of Form III crystalline atorvastatin calcium while PCT application W097/03959 teaches a method for the preparation of Form I, II, and IV of crystalline atorvastatin calcium.

PCT application WO 01/36384 discloses Form V of atorvastatin calcium. All of these patents/applications claim advantages over the existing forms of atorvastatin in various ways.

The differences in the physical properties of polymorphs result from the orientation and intermolecular interactions of adjacent molecules (complexes) in the bulk solid. Accordingly, polymorphs are distinct solids sharing the same molecular formula, which may be thought of as analogous to a unit cell in metallurgy, yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient, as well as their stability, a property which is very relevant in the case of Atorvastatin. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

International Patent Application PCT/IL05/00539 to some of the applicants of the present invention teaches a delayed burst release oral formulation for localized release of a statin in the GI tract. That formulation comprises a core comprising a statin and a burst controlling agent and an outer coating comprising a water insoluble hydrophobic carrier and a water insoluble hydrophilic particulate matter. The particulate matter, which allows entry of liquid into the core, is preferably a hydrophilic yet water insoluble polymer.

Statins, including atorvastatin, are sensitive to environmental pH, oxygen, light, temperature, humidity, carbon dioxide and certain incompatible excipients. Such “incompatible excipients” are pharmaceutically acceptable excipients that are not suitable for use in a formulation with Atorvastatin; they include but are not limited to any excipient that may form any undesirable complex or undergo a chemical reaction with atorvastatin, for example by removing the Ca²⁺ (calcium) ion from atorvastatin calcium and causing it to convert to the lactone form through destabilization; or alternatively excipients which create an acidic environment. These incompatible excipients react with Atorvastatin during the production process and/or during storage and degrade it to produce impurities. The presently marketed commercial product, Lipitor® (Pfizer), contains Atorvastatin calcium and requires a stabilizer, such as CaCO₃. Stabilization techniques already known in the art are listed below, all of which are hereby incorporated by reference as if fully set forth herein.

The following patents or patent applications WO 02/072073 (Lek); WO9416693 (Warner-Lambert); U.S. Pat. No. 5,686,104 (Warner-Lambert); U.S. Pat. No. 6,126,971 (Warner-Lambert), EP 0680320 (Warner-Lambert); WO 01/93860 (Lek); and WO 00/35425 (Lek) discuss stabilization of atorvastatin and more particularly its hemi calcium salt with alkaline agents, buffering compounds or basifying agents.

WO 01/093859 (Lek) suggests stabilization of statin formulations by adding a substance capable of binding and/or neutralizing carbon oxide

WO 02/089788 (Biochemie) suggests amino sugars for stabilization of atorvastatin.

WO 04/071403 (Lek) relates to coated particles protecting the active agent atorvastatin from environmental influences.

WO 01/6566 (Teva) discloses stabilization of atorvastatin by polymers comprising at least one amino group or at least one amido group

WO 04/032920 (Lek) describes stabilization of amorphous atorvastatin exposed to an inert gas atmosphere.

WO 04/071402 (Lek) describes stabilization of statins by reducing the water content in the formulation or by stabilizing them with different types of microcrystalline cellulose and/or colloidal SiO₂.

The above art describes stable Atorvastatin cores formulation based on the use of stabilizers such as CaCO₃, alkaline and earth alkaline ions salts, alkalinizing and buffering agents, Crospovidone and so forth, as described above. Other solutions relate to reducing the amount of water in the formulation, which is both inconvenient and also difficult to achieve for long term stability. In the case of WO 04/032920, stabilization through special processing is suggested, by placing amorphous atorvastatin in an inert gas atmosphere. All of these approaches have a number of clear drawbacks, as they require special formulations and/or processing to be effective, which is both expensive and inconvenient.

### SUMMARY OF THE INVENTION

The background art does not teach or suggest a stable pharmaceutical formulation comprising atorvastatin and salts or other pharmaceutically acceptable thereof using only conventional pharmaceutical excipients.

The present invention overcomes these deficiencies of the background art by providing formulations, methods of use thereof and methods of manufacture thereof which are simple and efficient to produce, which provide good formulation stability and bioefficacy and which can provide any kind of fast or controlled release and thus suitable pharmacokinetics for atorvastatin. The formulations may optionally be free of a stabilizer such as CaCO₃ and also are preferably free of incompatible excipients such as croscarmellose sodium, carmellose calcium and sodium starch glycolate, which were shown to have deleterious effects on the active ingredient. More preferably, preferred embodiments of formulations according to the present invention comprise starch or pregelatinized starch (preferably pregelatinized starch like starch 1500) and/or lactose (preferably lactose monohydrate), The formulation of the present invention comprises atorvastatin or salts thereof in amorphous or any known crystal form and remains stable to the environmental influences optionally without addition of any stabilizers, such as alkalizing agents, buffering agents, etc., and only by using totally conventional excipients which are as compatible as possible with atorvastatin and salts thereof. However, optionally and preferably, one or more of the formulation and/or the form of the active ingredient is adjusted in order to provide greater stability and/or bioefficacy for the formulation according to the present invention.

Optionally and more preferably the formulation contains: amorphous or crystalline atorvastatin calcium as an active ingredient; starch and/or pregelatinized starch and/or Lactose as compatible major excipients; optionally and preferably compatible minor excipients such as (but not limited to) silicon dioxide, microcrystalline cellulose, HPC, HPMC, PVP, Crospovidone, Tween, Magnesium stearate; optionally incompatible excipents such as Croscarmellose sodium, carmellose calcium, sodium starch glycolate and stearic acid are preferably absent or if present, are in sufficiently low quantities so as to be unable to influence the active ingredient stability. With regard to incompatible excipients, the amount depends upon such factors as whether they are processed with the active ingredient during a wet or dry process and also with regard to the temperature to which the formulation is exposed during this processing. If a wet process is used, such as wet granulation for example, preferably these incompatible excipients are not used at least during the wet portion of such processing, and if used, are preferably present in an amount of only up to about 10% or even less depending on the degree of incompatibility.

The formulation according to the present invention provides the same good results in terms of stability as the formulation in which a known stabilizer such as CaCO₃ is used even if such a stabilizer is not present in the formulation and/or is present in an amount much lower than that which is known in the art.

The present invention relates to a new formulation which is stable without using any stabilizer by selecting suitable excipients which are inert to Atorvastatin.

The active ingredient in the formulations and methods of the present invention comprises atorvastatin and optionally its pharmaceutically acceptable salts thereof. Different crystal and amorphous forms of atorvastatin and pharmaceutically acceptable salts thereof have been described. The present invention also comprises such crystal and amorphous forms.

An optional but preferred form of atorvastatin is atorvastatin calcium, preferably with one or more than one excipient that is selected from the group consisting of lactose (preferably lactose monohydrate), starch (preferably pregelatinized starch such as starch 1500) or regular starch.

Optionally and preferably the formulation comprises at least one minor excipient being compatible with Atorvastatin or a pharmaceutical acceptable salt thereof such as (but not limited to) silicon dioxide, microcrystalline cellulose, HPC, HPMC, PVP, Crospovidone, Tween®, Magnesium stearate.

Optionally, the formulation comprises at least one minor excipient not being compatible with Atorvastatin or a pharmaceutical acceptable salt thereof (such as Croscarmellose, sodium starch glycolate, Carmellose calcium and Stearic acid and so forth), preferably used in a sufficiently low amount and/or processed with Atorvastatin in a dry and low temperature process (such as dry granulation or dry mixing at a low temperature), so as not to react with Atorvastatin. Preferably, the amount is adjusted according to whether a crystalline or amorphous form of Atorvastatin is used. For example, a lower amount of incompatible excipient is preferably used in a formulation containing amorphous form of atorvastatin, as the amorphous form is known to be less stable.

The minor excipients referred to above are selected but not limited to the following family of excipients: a filler, a tabletting aid, a flow regulating agent, a hardness enhancer, a glidant, a lubricant, an absorption enhancer, a binder, a disintegrant, and optionally at least one other excipient or a combination thereof.

Examples of a binder include but are not limited to Povidone (PVP: polyvinyl pyrrolidone), low molecular weight HPC (hydroxypropyl cellulose), low molecular weight HPMC (hydroxypropyl methylcellulose), low molecular weight carboxymethyl cellulose, ethylcellulose, gelatin, polyethylene oxide, acacia, dextrin, magnesium aluminum silicate, starch, and polymethacrylates. More preferably, the binder is HPC or Povidone.

Examples of a disintegrant include but are not limited to, Crospovidone (cross-linked PVP), pregelatinized starch (such as starch 1500 for example), microcrystalline starch, water insoluble starch, calcium carboxymethyl cellulose, magnesium aluminum silicate (Veegum) or a combination thereof. Most preferably, the disintegrant is pregelatinized starch.

Examples of suitable fillers include but are not limited to, microcrystalline cellulose (e.g., Avicel®), starch, lactitol, lactose, dibasic calcium phosphate or any other type of suitable inorganic calcium salt and sucrose, or a combination thereof. A preferred filler is lactose monohydrate.

Examples of suitable lubricants include but are not limited to, stearate salts such as magnesium stearate, calcium stearate, and sodium stearate; stearic acid, talc, sodium stearyl fumarate, and compritol (glycerol behenate), corola oil, glyceryl palmitostearate, hydrogenated vegetable oil, magnesium oxide, mineral oil, poloxamer, polyethylene glycol, polyvinyl alcohol, sodium benzoate, talc, sodium stearyl fumarate, compritol (glycerol behenate) and sodium lauryl sulfate (SLS) or a combination thereof. A currently preferred lubricant is magnesium stearate.

Examples of suitable flow regulating agents include but are not limited to, colloidal silicon dioxide and aluminum silicate. A currently preferred flow regulating agent is colloidal silicon dioxide.

Examples of suitable hardness enhancer include but are not limited to silicon dioxide which is known to improve hardness of pregelatinized starch containing tablets.

The core can also optionally include a buffering agent such as, for example, an inorganic salt compound and an organic alkaline salt compound. Preferably, the buffering agent is selected from the group consisting of potassium bicarbonate, potassium citrate, potassium hydroxide, sodium bicarbonate, sodium citrate, sodium hydroxide, calcium carbonate, dibasic sodium phosphate, monosodium glutamate, tribasic calcium phosphate, monoethanolamine, diethanolamine, triethanolamine, citric acid monohydrate, lactic acid, propionic acid, tartaric acid, fumaric acid, malic acid, and monobasic sodium phosphate.

The core can also optionally contain at least one of a wetting agent, suspending agent, surfactant, and dispersing agent, or a combination thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, docusate sodium.

Examples of suitable suspending agents include but are not limited to, alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, and tragacanth.

Examples of suitable surfactants include but are not limited to, anionic surfactants such as docusate sodium and sodium lauryl sulfate; cationic, such as cetrimide; nonionic, such as polyoxyethylene sorbitan fatty acid esters (polysorbates) and sorbitan fatty acid esters.

Examples of suitable dispersing agents include but are not limited to, poloxamer, polyoxyethylene sorbitan fatty acid esters (polysorbates) and sorbitan fatty acid esters.

The content of the wetting agent, surfactant, dispersing agent and suspending agent can range in an amount of from about 0% to about 30% of the weight of the formulation, although preferably they are present in an amount of from about 0 to about 10%. The outer coating or the core or both can also optionally contain at least one of a wetting agent, suspending agent, surfactant, and dispersing agent, or a combination thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, docusate sodium.

Examples of suitable suspending agents include but are not limited to, alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, and tragacanth.

Examples of suitable surfactants include but are not limited to, anionic surfactants such as docusate sodium and sodium lauryl sulfate; cationic, such as cetrimide; nonionic, such as polyoxyethylene sorbitan fatty acid esters (polysorbates) and sorbitan fatty acid esters.

Examples of suitable dispersing agents include but are not limited to, poloxamer, polyoxyethylene sorbitan fatty acid esters (polysorbates) and sorbitan fatty acid esters.

According to preferred embodiments of the present invention, there is provided a pharmaceutical formulation of atorvastatin or any acceptable salt thereof free of any stabilizer. According to preferred embodiments of the present invention, there is provided a modified release pharmaceutical formulation of atorvastatin free from any stabilizer.

According to preferred embodiments of the present invention, there is provided a formulation comprising a core containing atorvastatin and a release controlling agent. Optionally and preferably, the release controlling agent is selected from the group consisting of methylcellulose, carboxymethylcellulose, hydroxypropylcellulose, hydroxypropylmethylcellulose, hydroxyethylcellulose; vinyl polymers; acrylic polymers and copolymers; natural and synthetic gums; gelatin, collagen, proteins, polysaccharides; and mixtures thereof. More preferably, the release controlling agent is hydroxypropylmethylcellulose.

Optionally and preferably, the release controlling agent comprises a vinyl polymer selected from the group consisting of polyvinylpyrrolidone, and polyvinyl alcohol.

Also optionally and preferably, the release controlling agent comprises acrylic polymers and copolymers selected from the group consisting of acrylic acid polymer, methacrylic acid copolymers, ethyl acrylate-methyl methacrylate copolymers.

Also optionally and preferably, the release controlling agent comprises gums selected from the group consisting of guar gum, arabic gum, xanthan gum.

Also optionally and preferably, the release controlling agent comprises a polysaccharide selected from the group consisting of pectin, pectic acid, alginic acid, sodium alginate, polyaminoacids, polyalcohols, polyglycols.

According to preferred embodiments of the present invention, the formulation optionally and preferably further comprises a coating for providing one of modified release, delayed release, controlled release, slow release, sustained release, extended release, delayed controlled or sustained release, or extended release, delayed burst release, delayed fast or rapid release of Atorvastatin.

More preferably, the coating provides a Time Controlled Delivery System (TCDS®) for atorvastatin.

According to preferred embodiments of the present invention, there is provided a formulation as described herein that releases atorvastatin or any pharmaceutical accepted salt thereof as active ingredient in the lower gastrointestinal tract of a subject.

Alternatively, the formulation releases atorvastatin or any pharmaceutical accepted salt thereof as active ingredient, in the small intestine of a subject.

Also alternatively, the formulation releases atorvastatin or any pharmaceutical accepted salt thereof as active ingredient, in the colon of a subject.

Optionally and preferably, the formulation comprises a core containing atorvastatin as described herein, coated with a coating for providing one of modified release, delayed release, controlled release, slow release, sustained release, extended release, delayed controlled or sustained release, or extended release, delayed burst release, delayed fast or rapid release of atorvastatin.

More preferably, the coating provides a Time Controlled Delivery System (TCDS®) for Atorvastatin as described herein.

According to preferred embodiments of the present invention, there is provided a formulation as described herein for providing an increased blood concentration of atorvastatin or any pharmaceutical accepted salt thereof as active ingredient, relative to that resulting from the administration of an equivalent dose of the conventional immediate release formulations.

Optionally and preferably, the formulation comprises a core containing atorvastatin as described herein, coated with a coating for providing one of modified release, delayed release, controlled release, slow release, sustained release, extended release, delayed controlled or sustained release, or extended release, delayed burst release, delayed fast or rapid release of atorvastatin.

More preferably, the coating provides a Time Controlled Delivery System (TCDS®) for Atorvastatin as described herein.

According to preferred embodiments of the present invention, there is provided a formulation as described herein that features a lower dose of atorvastatin or any pharmaceutical accepted salt thereof as active ingredient, relative to the conventional immediate release formulations. By “lower dose” it is meant that the formulation contains a reduced dose of atorvastatin, as compared with the corresponding conventional formulation, preferably up to about 60% of the conventional dose for atorvastatin.

Optionally and preferably, the formulation comprises a core containing atorvastatin as described herein, coated with a coating for providing one of modified release, delayed release, controlled release, slow release, sustained release, extended release, delayed controlled or sustained release, or extended release, delayed burst release, delayed fast or rapid release of atorvastatin.

More preferably, the coating provides a Time Controlled Delivery System (TCDS®) for Atorvastatin as described herein.

According to preferred embodiments of the present invention, there is provided a formulation as described herein that features a relatively lower dose of atorvastatin or any pharmaceutical accepted salt thereof as active ingredient, for providing an increased blood concentration of the said active ingredient, relative to that resulting from the administration of an equivalent dose of the conventional immediate release formulations. By “relatively lower dose” it is meant a dose that provides at least the same or similar pharmaceutical and/or therapeutic effect (if not a greater effect) as a conventional dose of atorvastatin, while featuring a lower amount of atorvastatin than the conventional dose of atorvastatin.

Optionally and preferably, the formulation comprises a core containing atorvastatin as described herein, coated with a coating for providing one of modified release, delayed release, controlled release, slow release, sustained release, extended release, delayed controlled or sustained release, or extended release, delayed burst release, delayed fast or rapid release of atorvastatin.

More preferably, the coating provides a Time Controlled Delivery System (TCDS®) for Atorvastatin as described herein.

According to other preferred embodiments of the present invention, there is provided a method for producing a stable pharmaceutical formulation comprising atorvastatin or salts thereof as active ingredient, the method comprising wet granulating atorvastatin with the proviso that the formulation is essentially free of croscarmellose or microcrystalline cellulose or any mono and/or di and/or tri valent metal containing excipients during the wet steps of the production process. Examples of such mono and/or di and/or tri valent metal containing excipients include but are not limited to sodium carboxymethyl cellulose, calcium carboxymethyl cellulose, sodium lauryl sulphate, calcium pectinate, sodium alginate, mono and di basic sodium phosphate, di and tri basic calcium phosphate, and sodium starch glycolate. Preferably, the formulation is essentially free of a stabilizer. More preferably, the formulation is essentially free of CaCO₃. More preferably, the formulation further comprises at least one major excipient in an amount of at least about 30%, wherein said at least one major excipient is granulated with said atorvastatin. Most preferably, the at least one major excipient comprises one or more of starch, pregelatinized starch or lactose.

According to other preferred embodiments of the present invention, there is provided a method for producing a stable pharmaceutical formulation comprising atorvastatin or salts thereof as active ingredient, the method comprising granulating atorvastatin with at least one major excipient comprising one or more of starch, pregelatinized starch or lactose.

Preferably, granulating comprises wet granulating.

According to other preferred embodiments of the present invention, there is provided a method for producing a stable pharmaceutical formulation comprising atorvastatin or salts thereof as active ingredient, the method comprising: wet granulating atorvastatin with at least one excipient, wherein said at least one excipient is free of an incompatible excipient to form a granulate; and after said wet granulation, adding an incompatible excipient to said granulate.

Optionally and preferably, the incompatible excipient is selected from the group consisting of Croscarmellose sodium, Carmellose Calcium, or sodium starch glycolate. More preferably, the minor incompatible excipient is present in an amount of up to about 10%. Most preferably, an amount of said minor incompatible excipient is determined according to a form of said atorvastatin. Optionally and preferably, the form of atorvastatin is determined according to one or more of a salt, a crystalline form or an amorphous form, alone or in combination. Preferably, atorvastatin comprises an atorvastatin salt. More preferably, atorvastatin salt comprises an alkaline earth metal. Also more preferably, the alkaline earth metal comprises calcium or magnesium. Most preferably, the atorvastatin salt comprises atorvastatin calcium. Most preferably, atorvastatin comprises crystalline atorvastatin calcium form VI as an active ingredient. Also most preferably, atorvastatin comprises amorphous atorvastatin as an active ingredient.

According to preferred embodiments of methods of preparing the formulation, the formulation is essentially free of any stabilizer. Optionally, the method further comprises forming a core from said wet granulate; and coating said core. Preferably, the method further comprises placing said core in a capsule. More preferably, the method further comprises packaging said core in a moisture sealed package. Most preferably, the moisture sealed package comprises an Alu/Alu package.

Optionally and alternatively, the method further comprises forming a core from said wet granulate; and placing said core in a capsule. More preferably, the method further comprises packaging said capsule in a moisture sealed package. Most preferably, the moisture sealed package comprises an Alu/Alu package.

Optionally and preferably, at least one excipient comprises one or more of starch, pregelatinized starch or lactose.

Also optionally and preferably, atorvastatin is micronized before wet granulation.

Preferably, the granulate is dried at a temperature up to about 60° C. before said at least one incompatible excipient is added. Also preferably, the wet granulation is performed with an aqueous granulation solution. More preferably, the aqueous granulation solution is free of any alcohol.

According to other preferred embodiments of the present invention, there is provided a stable formulation comprising atorvastatin and at least one major excipient in an amount sufficient to stabilize said atorvastatin, wherein said at least one major excipient is selected from the group consisting of lactose, starch and pregelatinized starch, wherein stability is determined according to the following criteria: after six months at 40° C./75% RH, a maximum known impurity selected from desfluoro or lactone is less than about 0.5%; a maximum level of any other impurity is less than about 0.5%; and total impurities are less than about 1.5%.

Preferably, an amount of said major excipient is determined according to a form of said atorvastatin. Optionally, the form of atorvastatin is determined according to one or more of a salt, a crystalline form or an amorphous form, alone or in combination. Preferably, atorvastatin comprises an atorvastatin salt. More preferably, atorvastatin salt comprises an alkaline earth metal. Also more preferably, the alkaline earth metal comprises calcium or magnesium. Most preferably, the atorvastatin salt comprises atorvastatin calcium. Most preferably, atorvastatin comprises crystalline atorvastatin calcium form VI as an active ingredient. Also most preferably, atorvastatin comprises amorphous atorvastatin as an active ingredient.

According to other preferred embodiments of the present invention, there is provided a stable formulation, comprising crystalline Atorvastatin calcium form VI with one or more of Lactose, starch and pregelatinized starch, free of Croscarmellose sodium, Carmellose calcium, Sodium starch glycolate or Stearic acid. Preferably, the formulation further comprises a binder selected from the group consisting of HPC, HPMC and PVP; Crospovidone, Tween®, magnesium stearate; Aerosil®, microcrystalline cellulose and Mannitol.

According to other preferred embodiments of the present invention, there is provided a stable formulation, comprising amorphous Atorvastatin calcium with one or more of Lactose, starch and pregelatinized starch, free of Croscarmellose sodium, Carmellose calcium, Sodium starch glycolate or Stearic acid.

Preferably, the formulation further comprises a binder selected from the group consisting of HPC, HPMC and PVP; Crospovidone, Tween®, magnesium stearate (lubricant); Aerosil®, microcrystalline cellulose and mannitol.

Unless otherwise indicated, all percentages of ingredients in formulations are weight by weight percent. Also unless otherwise indicated, all percentages of ingredients are given weight by weight percent separately for the core and for the coating (eg an ingredient in the core is given weight by weight percent for the core alone).

### BRIEF DESCRIPTION OF THE DRAWINGS

The invention is herein described, by way of example only, with reference to the accompanying drawings, wherein:

FIG. 1 shows the dissolution release profile in IF (intestinal fluid) pH 6.8 for the Amorphous Atorvastatin Calcium core #1 containing 30% starch 1500 and 62% lactose monohydrate (uncoated);

FIG. 2 shows the dissolution release profile in IF (intestinal fluid) pH 6.8 for the amorphous atorvastatin calcium core #2 containing 70% starch 1500 and 22% lactose monohydrate;

FIG. 3 shows the dissolution release profile in IF (intestinal fluid) pH 6.8 for the Crystalline form VI Atorvastatin Calcium Core #3, comprising 30% Starch 1500 and 62% lactose monohydrate;

FIG. 4 shows the dissolution release profile in IF (intestinal fluid) pH 6.8 for the Crystalline form VI Atorvastatin Calcium core #4, comprising 70% Starch 1500 and 22% lactose monohydrate.

### DESCRIPTION OF PREFERRED EMBODIMENTS OF THE INVENTION

According to preferred embodiments of the present invention, an atorvastatin formulation according to the present invention is preferably prepared with at least one excipient selected according to a form of atorvastatin, such as a crystalline form, an amorphous form, a salt or an acid of the base. It should be noted that the acid form is not currently commercially available, possibly due to its instability. More preferably, the form of atorvastatin is selected from the group consisting of crystalline form VI or amorphous, preferably as a salt although optionally the acid form may be used. More preferably, the salt is an alkaline earth metal hemi salt of Atorvastatin, which more preferably comprises either the magnesium or calcium salts; most preferably the salt is the calcium salt. Most preferably, the form of atorvastatin is either crystalline form VI calcium salt or amorphous calcium salt. The most preferred form of the calcium salt is the hemi-hydrate.

According to preferred embodiments of the present invention, the formulation comprises at least one excipient selected from the group consisting of lactose, starch, pregelatinized starch or a combination thereof. Preferably, such an excipient is a major excipient. Optionally, the formulation comprises at least about 30% weight per weight of the major excipient (or combination thereof), preferably at least about 50% weight per weight, more preferably at least about 70% weight per weight and most preferably at least about 90% weight per weight.

Preferably, the formulation comprises Atorvastatin with Lactose and Starch as major excipients. Optionally, lactose is present in an amount of up to about 90% weight per weight; when present in a mixture with at least one other major excipient, the amount of lactose may range from above 0% to below 90% of the formulation. Lactose may optionally be absent, in which case the amount is 0%. Also optionally, starch, preferably pregelatinized starch such as starch 1500 for example, is present in an amount of up to about 90% weight per weight; when present in a mixture with at least one other major excipient, the amount of starch may range from above 0% to below 90% of the formulation. Starch may optionally be absent, in which case the amount is 0%. Optionally and preferably, atorvastatin comprises the calcium salt, more preferably as either crystalline or amorphous atorvastatin, optionally as the hemi Magnesium salt or other salts or atorvasatin acid. Most preferably, the crystalline form is crystalline form VI. Optionally and preferably, atorvastatin is present in an amount of from about 1% to about 50% weight per weight according to the weight of the base, preferably from about 1 to about 30%, more preferably from about 1 to about 20% and most preferably from about 1 to about 10%.

Optionally the formulation of the present invention comprises at least a minor compatible excipient. Preferably, the maximum combined amount of such minor compatible excipient(s) is up to about 50%, while for combined minor excipients, each such excipient is preferably present in an amount of from about 0% to about 35% weight per weight of the formulation.

According to preferred embodiments of the present invention, the minor compatible excipient is selected from the group consisting of a tabletting aid such as Aerosil®, preferably present in an amount of up to about 2%, crospovidone as superdisintegrant or disintegrant (preferably present in an amount of up to about 15%), mannitol as a filler (preferably present in an amount of up to about 35%), microcrystalline cellulose as a filler (for example Avicel) (preferably present in an amount of up to about 35%), PVP or HPC or HPMC as binders or hydrogel forming excipients (preferably present in an amount of up to about 20%), Talc as a glidant (preferably present in an amount of up to about 2%), Tween® as a surfactant (preferably present in an amount of up to about 2%), magnesium Stearate as a lubricant (preferably present in an amount of up to about 2%) or a combination thereof. The amounts of these minor compatible excipients are preferably determined according to the type of Atorvastatin used and are also preferably determined according to the type of process used. For example, since crystalline atorvastatin is more stable than amorphous atorvastatin, and since the calcium salt is the preferred form of atorvastatin, then optionally more microcrystalline cellulose could be added to a formulation comprising crystalline atorvastatin calcium (particularly for form VI) than for amorphous atorvastatin calcium. In terms of processing, preferably microcrystalline cellulose is not incorporated during wet processing, as in the wet stage of wet granulation; however, a small amount could optionally be used even during the wet stage of such processing if the atorvastatin comprised atorvastatin calcium.

According to other preferred embodiments of the present invention, the formulation comprises one or more than one minor incompatible excipient such as Croscarmellose sodium (superdisintegrant) [preferably present in an amount of from about 0 to about 10%] (preferably extragranular), Carmellose calcium (superdisintegrant) [preferably present in an amount of from about 0 to about 10%] (preferably extragranular), Sodium starch glycolate (superdisintegrant) [preferably present in an amount of from about 0 to about 10%] (preferably extragranular) preferably determined according to the type of Atorvastatin used as previously described. By “extragranular” it is meant that the excipient is preferably not added to the formulation during granulation, particularly for wet granulation.

According to other preferred embodiments of the present invention, the formulation comprises a core, the core comprising atorvastatin and at least one major excipient as described above, optionally with at least one minor excipient, which is then coated with a coating. Any suitable coating which is known in the art may optionally be used, although preferably the coating provides a good seal to protect the core. Non limiting examples of coating materials include any suitable enteric polymer or polymer combination (as for that present in Opadry® (Colorcon Inc) or, Eudragit L or L30D, or S (Rohm Pharma)) and so forth. The formulation may optionally feature a fast or slow release inner core further coated with a Time Controlled Delivery System (TCDS®). Examples of such TCDS systems include but are not limited to, U.S. Pat. Nos. 6,531,152 and 5,840,332 by at least one of the present inventors, hereby incorporated by reference as if fully set forth herein.

However, the formulation may also optionally feature coated or uncoated cores or a granulate placed in a capsule such as a gelatin capsule for example, which may optionally be a soft or hard gelatin capsule.

According to preferred embodiments of the present invention, the formulation is prepared according to wet granulation, more preferably with an aqueous granulation solution. The wet granulation is then dried. Drying may optionally occur at temperatures up to about 60° C. The granulate is optionally further mixed with extragranular excipients and then further processed according to one of the following methods: compressed to form tablets, optionally followed by coating and/or being placed in a capsule, such as a gelatin capsule (hard or soft) for example; or placed as a blend directly in the capsules. The tablets or capsules are preferably then packed in packaging that presents an effective barrier to moisture, such as Alu/Alu packaging for example. Optionally and preferably, the method features producing a stable pharmaceutical formulation comprising atorvastatin or salts thereof as active ingredient, by wet granulating atorvastatin with the proviso that the formulation is essentially free of a stabilizer. Preferably, the formulation is essentially free of CaCO₃. Optionally and preferably, the active ingredient is micronized before granulation.

Results provided below through experimental testing indicate that the preferred embodiments of the formulation according to the present invention assures the stability of atorvastatin, even when the formulation is wet granulated and dried for many hours at high temperatures such as 60° C. as usually done in the common state of the art, especially when the active component has poor solubility and must be used as a micronized powder with low flow and poor mixing properties.

The formulation may optionally be implemented as a fast release coated or uncoated tablet whose in vitro properties are exactly the same as Lipitor® as far as dissolution profile (in any medium tested), disintegration time, assay and stability are concerned. This probably means that such a tablet would be bioequivalent to Lipitor®. The dissolution profile, stability and other physicochemical properties of this formulation according to the present invention are little influenced by the granulation, drying and tabletting equipment and parameters used for its production. It is also stable even with a wide range of Starch (preferably pregelatinized starch)/Lactose ratios in the formula. Preferably such a ratio ranges from about 5%/95% to about 95%/5%.

### EXAMPLES

The Examples given below are intended only as illustrations of various embodiments of the present invention, and are not intended to be limiting in any way.

### Section I: Description of the Analytical Methods

As described in greater detail below, a number of analytical methods were used for the experiments described in Sections II and III below. A description of these methods is provided herein.

Loss On Drying (LOD): LOD is a method for determining the amount of water or humidity in the formulation. This method is based on the weight which is lost during a heating process, at a relatively high temperature, of a sample. LOD of granulates or of crushed tablets was checked according to the gravimetric method using a LP16 Mettler IR dryer. The test parameters were as follows: sample weight from 3-5g—drying temperature 110° C.—end of the test: sample weight decrease is not more than 2 mg between 2 weighing separated by 2 minutes.

Dissolution test: The dissolution tests of the cores or coated tablets were performed in USP apparatus II fitted with paddles, at 50 rpm and 37° C. The dissolution media were either 0.1N Hydrochloric acid or 0.05M buffer Phosphate such as pH 6.8, 4.5 and others, with various concentrations of surface active agents like polysorbate 80. The release was determined using a Waters liquid chromatograph equipped with a UV detector operating at a wavelength of 238 nm. The column was a Hypersil BDS (4.6 mm×3 cm) 3-μm column. The mobile phase was composed of a 55:45 mixture of 0.1% Phosphoric acid in water:acetonitrile. The injection volume was 20 μL, and the flow rate was 2.5 mL/min. The atorvastatin retention time is about 1 min.

The standards concentration set was 11.1, 22.2 and 44.4 ppm for 10, 20 and 40 mg tablets respectively, made in a water:methanol diluent.

Assay and impurities tests: The tests were performed on a Waters liquid chromatograph equipped with a UV detector operating at a wavelength of 238 nm. The column was a Purospher RP-18e (4.0 mm×15 cm) 5 -μm column. The mobile phase was composed of a 55:45 mixture of 0.1% Phosphoric acid in water:acetonitrile. The injection volume was 20 μL, and the flow rate was 1.0 mL/min. The atorvastatin retention time is about 10 min.

The standards and sample concentrations of the assay is about 200 ppm. The standard for the related compounds is about 2 ppm (0.2% of the sample concentration), made in a water:methanol diluent. Results of related compounds were expressed as a percentage of the total amount of atorvastatin calcium in the sample. Unknown impurities were named according to the relative retention time according to the method.

Disintegration test: The disintegration time of cores or coated tablets were measured according to the USP method without disk either in 0.1 M HCl or 0.05 M phosphate buffer PH=6.8.

### Section II: Compatibility Testing

Compatibility tests were performed according to the following procedure, in order to ensure that the presence of any individual excipient in a mixture with the drug substance does not induce the formation of impurities, cause instability or otherwise have a harmful influence. These experiments showed that, surprisingly, preferred embodiments of the formulation according to the present invention had good stability characteristics without a stabilizer, and also that the selected excipients according to the present invention had good compatibility characteristics with the active ingredient.

In order for an excipient to be used with an embodiment of the formulation of the present invention, it was required to perform acceptably during the compatibility testing. The acceptance criteria for the compatibility test were as follows. The results of the impurity levels of the mixtures of the Atorvastatin calcium drug substance with the tested excipients should be similar to the results of the impurity levels of the Atorvastatin calcium drug substance sample, which is the active ingredient alone, such that the addition of one or more excipients does not adversely affect the drug itself, leading to an increase in impurities or a lack of physical stability. Physical stability was determined by examining the mixture's appearance in terms of discoloration, liquefaction, dryness and odor or gas.

Experimental Procedure

A granulate or dry mix of the drug substance and each of the excipients requested to the expected ratio in the possible final formulas was prepared. The granulate was prepared manually with a mortar and pestle. The active ingredient is mixed with the ingredient(s) to be tested, then granulated in a mortar and pestle using the aqueous granulation solution. The wet granulate was then dried in an oven at 60° C. down to LOD<5% and then milled.

Each sample contained a final weight of about 1 gr.

The calculated weights for 1 gr dry granulate or dry blend is as follows.

TABLE 1A
The calculated weight for each vial for crystalline form VI

| | | | |
|---|---|---|---|
| | | | Weight of |
| | Lot #/ | Weight of | Atorvastatin |
| Ingredients | Entry # | excipient (mg) | (mg) |

Compatibility with crystalline form VI Atorvastatin Ca (granulates)

| | | | | |
|---|---|---|---|---|
| Vial 1 | Atorvastatin | 1201 | — | 1000 |
| Vial 2 | Lactose monohydrate | 1111 | 833 | 167 |
| Vial 3 | CaCO₃ | SGTV | 833 | 167 |
| Vial 4 | Avicel ® 101 | 1244 | 833 | 167 |
| Vial 5 | Croscamellose sodium | 1162 | 833 | 167 |
| Vial 6 | Crospovidone | 1322 | 833 | 167 |
| Vial 7 | Starch 1500 | 1381 | 833 | 167 |
| Vial 8 | Na Starch glycolate | — | 833 | 167 |
| Vial 9 | Carmellose calcium | — | 833 | 167 |
| Vial 10 | Aerosil ® | — | 333 | 667 |
| Vial 11 | Stearic acid | — | 7 | 993 |

TABLE 1B
The calculated weight for each vial for the amorphous form
Compatibility with amorphous Atorvastatin Calcium (dry mixes)

| | | | Weight of | Weight of |
|---|---|---|---|---|
| | | Lot #/ | excipient | atorvastatin |
| | Ingredients | Entry # | (mg) | (mg) |
| Vial 1 | Atorvastatin | 1414 | — | 1000 |
| Vial 2 | Starch 1500 | 1381 | 600 | 400 |
| Vial 3 | Starch 1500 | 1381 | 900 | 100 |
| Vial 4 | Lactose Monohydrate | 1360 | 600 | 400 |
| Vial 5 | Lactose Monohydrate | 1360 | 900 | 100 |
| Vial 6 | MCC PH 101 | 1244 | 600 | 400 |
| Vial 7 | MCC PH 101 | 1244 | 900 | 100 |
| Vial 8 | MCC PH 102 | 1326 | 600 | 400 |
| Vial 9 | MCC PH 102 | 1326 | 900 | 100 |
| Vial 10 | CaCO₃ | LNK771 | 600 | 400 |
| Vial 11 | CaCO₃ | LNK771 | 900 | 100 |
| Vial 12 | Aerosil ® | — | 333 | 667 |
| Vial 13 | Mannitol | | 900 | 100 |
| Vial 14 | Stearic acid | — | 7 | 993 |
| Vial 15 | Isopropyl Alcohol (w/o | — | 167 | 833 |
| | water addition) | | | |
| Vial 16 | Ethanol | — | 167 | 833 |
| | (w/o water addition) | | | |

MCC: Microcrystalline cellulose

The compatibility test was performed as follows. Each mixture (blend or granulate) was transferred to a vial, 0.2 ml of purified water was added, and the mixtures were mixed with a Pasteur pipette, which was then broken and inserted in the vial in order to avoid any loss of material.

The vials were then sealed and stored at a temperature of 50° C. for two weeks.

After two weeks the vials were removed, examined for physical changes and were tested for impurities according to the HPLC method (RD 2000-1) as described in greater detail below.

A suitable calculated amount of each blend or granulate containing 20 mg of atorvastatin calcium was taken and transferred to a 100 ml volumetric flask. 75 ml of diluent was added and the samples were shaken for 30 min, and then diluted to volume with diluent.

5 ml of the above mentioned solution was transferred and diluted in a 10 ml volumetric flask. The diluent for all experiments was water:methanol mixture.

The samples were tested in reference to an external standard prepared by weighing 21.7 mg of atorvastatin calcium (raw material, unformulated) to a 100 ml volumetric flask to form a stock solution, then diluting the stock solution to 0.2%.

Results are summarized below.

TABLE 2A
Results of the compatibility tests for crystalline form VI
Compatibility with crystalline form VI Atorvastatin Ca (granulates)

| | | | Weight of | Weight of |
|---|---|---|---|---|
| | | | excipient | atorvastatin |
| | Ingredients | Compatibility | (mg) | (mg) |
| Vial 1 | Atorvastatin | Stable | — | 1000 |
| | | (reference) | | |
| Vial 2 | Lactose | Compatible | 833 | 167 |
| | monohydrate | | | |
| Vial 3 | CaCO₃ | Compatible | 833 | 167 |
| Vial 4 | Avicel 101 | almost | 833 | 167 |
| | | compatible | | |
| Vial 5 | Croscamellose | Very | 833 | 167 |
| | sodium | Incompatible | | |
| Vial 6 | Crospovidone | Compatible | 833 | 167 |
| Vial 7 | Starch 1500 | Compatible | 833 | 167 |
| Vial 8 | Na Starch glycolate | Incompatible | 833 | 167 |
| Vial 9 | Carmellose calcium | Incompatible | 833 | 167 |
| Vial 10 | Aerosil ® | Compatible | 333 | 667 |
| Vial 11 | Stearic acid | Incompatible | 70 | 930 |

TABLE 2B
Results of the compatibility tests for the amorphous form
Compatibility with amorphous Atorvastatin Ca (dry mixes)

| | | | Weight of | Weight of |
|---|---|---|---|---|
| | | | excipient | Atorvastatin |
| | Ingredients | Compatibility | (mg) | (mg) |
| Vial 1 | Atorvastatin | Stable | | |
| | | (reference) | | |
| Vial 2 | Starch 1500 | Compatible | — | — |
| Vial 3 | Starch 1500 | Compatible | 600 | 600 |
| Vial 4 | Lactose | Compatible | 900 | 900 |
| | Monohydrate | | | |
| Vial 5 | Lactose | Compatible | 600 | 600 |
| | Monohydrate | | | |
| Vial 6 | MCC PH 101 | Compatible | 900 | 900 |
| Vial 7 | MCC PH 101 | Compatible | 600 | 600 |
| Vial 8 | MCC PH 102 | Compatible | 900 | 900 |
| Vial 9 | MCC PH 102 | Compatible | 600 | 600 |
| Vial 10 | CaCO₃ | Compatible | 900 | 900 |
| Vial 11 | CaCO₃ | Compatible | 600 | 600 |
| Vial 12 | Mannitol | Almost | 900 | 100 |
| | | compatible | | |
| Vial 13 | Aerosil ® | Compatible | 333 | 667 |
| Vial 14 | Stearic acid | Incompatible | 70 | 930 |
| Vial 15 | Isopropyl Alcohol | Incompatible | 167 | 833 |
| | (w/o water) | | | |
| Vial 16 | Ethanol (w/o water) | Incompatible | 167 | 833 |

MCC = microcrystalline cellulose

As shown above, the inventors found that surprisingly, many excipients proved to be compatible with atorvastatin calcium and that, as a result, some stabilizer free solid dosage form formulation of atorvastatin (calcium) could be invented.

The inventors also found that surprisingly, sodium croscarmellose, which is used in the formulation of the innovator (Lipitor® by Pfizer) as a disintegrant, has an extraordinarily deleterious effect on Atorvastatin calcium. Without wishing to be limited by a single hypothesis, this may be why the original manufacturer had to add a large amount of stabilizer in their formula (22% of CaCO₃).

Other disintegrants tested also gave poor compatibility results except Crospovidone and pregelatinized starch such as Starch 1500 for example (which is known to be both a filler and a tablet disintegrant). Therefore preferred disintegrants according to the present invention comprise one or both of them.

### Section III: Testing of Formulations According to the Present Invention

### Details of the Experiments

Various further experiments were performed as described in greater detail below, in order to assess which excipients are preferably used with particular forms of atorvastatin (tested as atorvastatin calcium), for example with regard to whether the active ingredient is in a particular crystalline form or amorphous form. The term “almost compatible” means that the ingredient showed some compatibility with atorvastatin in the amount tested, but that compatibility could presumably be increased by lowering the amount of the ingredient in the final formulation, adding it to the formulation at particular stages in the process of production (for example, adding it after the wet stages of wet granulation), combining with one or more ingredients of greater compatibility, or a combination thereof.

Experiment 1 :Compatibility and Stability of Crystalline Atorvastatin Calcium Form VI Granulated with Various Excipients

This experiment tested which excipients are suitable to be formulated and preferably wet granulated with crystalline form VI atorvastatin calcium by wet granulating the active ingredient with the tested excipient and checking the stability of the active ingredient in the dry granulate both in a compatibility test 15 days at 50° C. at 16.7% LOD and 6 months in a stability test at 40 C/75% RH.

Details of the experiments: 3 g of crystalline Atorvastatin calcium from VI and 15 g of the excipient tested were granulated manually with mortar and pestle using between 3 and 5 ml granulation solution containing water, Klucel LF and Tween. The exact formula of each wet granulate is detailed below in Table 3, while the results of the stability testing are given in Table 4.

TABLE 3
Formulations of the wet granulates with crystalline atorvastatin calcium form VI

| | Atorvastatin | | | | | % LOD of |
|---|---|---|---|---|---|---|
| | calcium form | | Klucel | Tween ® | Water | the wet |
| Granulate | VI | Excipient | LF (g) | (g) | (g) | granulate |
| B | 3 g | 15 g lactose | 0.172 | 0.034 | 3 | ~17% |
| C | 3 g | 15 g CaCO₃ | 0.288 | 0.058 | 5 | ~20% |
| D | 3 g | 15 g Avicel | 0.288 | 0.058 | 5 | ~20% |
| | | PH101 | | | | |
| E | 3 g | 15 g | 0.288 | 0.058 | 5 | ~20% |
| | | Croscarmellose | | | | |
| | | Na | | | | |
| F | 3 g | 15 g crystalline | 0.288 | 0.058 | 5 | ~20% |
| (reference) | | atorvastatin | | | | |
| | | calcium from VI | | | | |
| G | 3 g | 15 g | 0.288 | 0.058 | 5 | ~20% |
| | | Crospovidone | | | | |
| H | 3 g | 15 g Starch 1500 | 0.288 | 0.058 | 5 | ~20% |
| I | 3 g | 15 g Na Starch | 0.288 | 0.058 | 5 | ~20% |
| | | glycolate | | | | |
| J | 3 g | 15 g Ca | 0.288 | 0.058 | 5 | ~20% |
| | | Carmellose | | | | |
| K* | 0.67 g | 0.33 g Aerosil ® | — | — | — | 0% |
| L* | 0.93 g | 0.07 g Stearic | — | — | — | 0% |
| | | acid | | | | |

*Aerosil ® and Stearic acid were not granulated and were tested in different ratios than other excipients because they are usually used in small quantities in common solid dosage form formulation.

The wet granulates were placed in oven at 50° C. for 1 or 2 days for drying, after which the dry granulates were sieved through a 600μ sieve and checked for LOD (loss on drying) as previously described.

Part of each dry granulate was placed in a 34 ml Securitainer® (a regular secure medicine bottle made from high density polyethylene; available from Jaycare Ltd in the United Kingdom) and placed in incubator at 40° C./75% RH for a 6 month stability study. Next, separate samples containing 1 g of each dry granulate were mixed with 200 μl water (LOD of the blend 16.67%), closed in a glass vial and stored at 50° C. for a 2 week compatibility test.

Results of those compatibility and stability studies are summarized in table 4; a more detailed description of the same results is provided in table 5 below. Stability or compatibility criteria were defined as: maximum known impurity <0.5% (preferably comprising one or both of the Desfluoro (Desfl.) or lactone (Lact.) degradation forms); maximum unknown impurity <0.5%, preferably <0.3%; total impurities <1.5%.

TABLE 4
Stability and compatibility of crystalline atorvastatin
calcium form VI with the different excipients tested

| | | | Stability at 40 C./ | |
|---|---|---|---|---|
| | | | 75 RH and LOD | Compatibility |
| Gran- | Excipient | LOD at | (checked at the end of | at 50° C. & |
| ulate | tested | t = 0 | the experiment) | 16.67% LOD |
| B | Lactose | 1.3% | Stable at least 6 | Compatible |
| | | | months. LOD 1.53% | |
| C | CaCO₃ | 0.5% | Stable at least 6 | Compatible |
| | | | months. LOD 0.83% | |
| D | Avicel PH101 | 2.78% | Stable only 2 months. | Compatible |
| | | | LOD 6.86% | |
| E | Croscarmellose | 5% | Unstable even at t = 0 | Not |
| | Na | | (2.35% lactone) | Compatible |
| | | | LOD 5.00% | |
| F | Reference w/o | 2.3% | Stable at least 6 | Compatible |
| | excipient | | months. LOD 3.03% | |
| G | Crospovidone | 4.3% | Stable at least 6 | Compatible |
| | | | months. LOD 16.17% | |
| H | Starch 1500 | 4.71% | Stable at least 6 | Compatible |
| | | | months. LOD 12.18% | |
| I | Na Starch | 5.29% | Unstable even at t = 0 | Not |
| | glycolate | | (0.9% lactone) | Compatible |
| | | | LOD 5.29% | |
| J | Ca Carmellose | 6.69% | Unstable even at t = 0 | Not |
| | | | (0.9% lactone) | Compatible |
| | | | LOD 6.69% | |
| K | Aerosil ® | NP | NP | Compatible |
| L | Stearic Acid | NP | NP | Incompatible |

TABLE 5
Stability and compatibility of crystalline Atorvastatin calcium form VI with the
different excipients tested (detailed presentation of the results)
Granulate B Lactose/Atorvastatin 5:1

| Stability 40 C./75% RH | | | Detailed impurities (RRT) | | | | |
|---|---|---|---|---|---|---|---|
| Time (mnths) | LOD | Total Imp | Desfl | Lact | 2.47 | 2.74 | |
| 0 | 1.3 | 0.19 | 0.19 | | | | Compatible 2 weeks |
| 0.5 | 1.65 | 0.54 | 0.23 | 0.15 | | 0.16 | 50° C./16.7% LOD |
| 1 | 1.63 | 0.3 | 0.2 | 0.13 | | | |
| 2 | 1.58 | 0.33 | 0.18 | 0.15 | | | |
| 3 | 1.57 | 0.34 | 0.1 | 0.24 | | | |
| 6 | 1.53 | 0.29 | <LOQ | 0.2 | | 0.1 | |

Granulate C CaCO₃/Atorvastatin 5:1

| Stability 40° C./75% RH | | | | | | | | |
|---|---|---|---|---|---|---|---|---|
| | | Total | Detailed impurities (RRT) | | | | | |
| Time (mths) | LOD | Imp | Desfl | Lact | 0.74 | 2.47 | 2.74 | |
| 0 | 0.5 | 0.22 | 0.22 | | | | | Compatible 2 weeks |
| 0.5 | 0.72 | 0.46 | 0.28 | | 0.06 | | 0.12 | 50° C./16.7% LOD |
| 1 | 0.80 | 0.17 | 0.17 | | | | | |
| 2 | 0.84 | | | | | | | |
| 3 | 0.84 | 0.12 | 0.12 | | | | | |
| 6 | 0.83 | 0.08 | <LOQ | | | | 0.08 | |

Granulate D Avicel/Atorvastatin 5:1

| Stability 40° C./75% RH | | | Detailed impurities (RRT) | | | | |
|---|---|---|---|---|---|---|---|
| Time (mths) | LOD | Total Imp | Desfl | Lact | 2.47 | 2.74 | |
| 0 | 2.78 | 0.33 | 0.19 | 0.14 | | | Compatible 2 weeks |
| 0.5 | 4.33 | 0.7 | 0.21 | 0.49 | | | 50° C./16.7% LOD |
| 1 | 5.15 | 0.65 | 0.18 | 0.47 | | | |
| 2 | 6.86 | 0.75 | 0.15 | 0.6 | | | |
| 3 | 7.14 | 0.81 | 0.09 | 0.71 | | | |
| 6 | 7.0 | | | | | | |

Granulate E Croscarmellose Na/Atorvastatin 5:1 (RRT)

| Stability 40 C./75% RH | | | | | | | | | |
|---|---|---|---|---|---|---|---|---|---|
| | | Total | Detailed impurities | | | | | | |
| Time (mths) | LOD | Imp | Desfl | Lact | 1.15 | 1.5 | 2.47 | 2.74 | |
| 0 | 5 | 2.56 | 0.21 | 2.35 | | | | | Not |
| 0.5 | 5.94 | 22.8 | 0.21 | 22.1 | 0.28 | 0.11 | | 0.16 | compatible |
| 1 | 6.66 | 4.45 | 0.17 | 4.3 | | | | | 50° C./16.7% |
| 2 | 11.59 | | | | | | | | LOD |
| 3 | 13.99 | | | | | | | | |
| 6 | | | | | | | | | |

Granulate F Atorvastatin calcium alone

| | | | Detailed | | | | |
|---|---|---|---|---|---|---|---|
| | | | impurities | | | | |
| Stability 40 C./75% RH | | | (RRT) | | | | |
| Time (mths) | LOD | Total Imp | Desfl | Lact | 2.47 | 2.74 | |
| 0 | 2.3 | 0.19 | 0.19 | | | | Compatible 2 weeks |
| 0.5 | 2.53 | 0.23 | 0.23 | | | | 50 C./16.7% LOD |
| 1 | 2.74 | 0.21 | 0.21 | | | | |
| 2 | 3.06 | 0.22 | 0.22 | | | | |
| 3 | 3.07 | 0.12 | 0.12 | | | | |
| 6 | 3.03 | 0.04 | 0.04 | | | | |

Granulate G Crospovidone/Atorvastatin 5:1

| | |
|---|---|
| Stability 40 C./75% RH | Detailed impurities (RRT) |

| Time (mths) | LOD | Total Imp | Desfl | Lact | 0.34 | 2.47 | 2.7 | |
|---|---|---|---|---|---|---|---|---|
| 0 | 4.3 | 0.19 | 0.19 | | | | | Compatible |
| 0.5 | 4.58 | 0.29 | 0.08 | 0.12 | 0.1 | | | 2 weeks |
| 1 | 4.88 | | | | | | | 50 C./16.7% |
| 2 | 5.78 | | | | | | | LOD |
| 3 | 7.62 | 0.31 | 0.11 | 0.09 | | 0.11 | 0.11 | |
| 6 | 16.17 | 0.5 | 0.12 | 0.08 | | 0.4 | 0.09 | |

Granulate H Starch 1500/Atorvastatin 5:1

| Stability 40° C./75% RH | | | Detailed impurities (RRT) | | | | |
|---|---|---|---|---|---|---|---|
| Time (mths) | LOD | Total Imp | Desfl | Lact | 2.47 | 2.74 | |
| 0 | 4.71 | 0.3 | 0.2 | 0.12 | | | Compatible 2 weeks |
| 0.5 | 5.28 | 0.35 | 0.09 | 0.26 | | | 50° C./16.7% LOD |
| 1 | 5.80 | 0.3 | 0.18 | 0.12 | | | |
| 2 | 11.17 | 0.35 | 0.1 | 0.24 | | | |
| 3 | 11.85 | 0.62 | 0.1 | 0.4 | | 0.13 | |
| 6 | 12.18 | 0.62 | 0.13 | 0.33 | | 0.17 | |

Granulate I Na Starch Glycolate/Atorvastatin 5:1

| | | | Detailed | | | | | |
|---|---|---|---|---|---|---|---|---|
| Stability 40° C./75% RH | | | impurities (RRT) | | | | | |
| Time (mths) | LOD | Total Imp | Desfl | Lact | 1.15 | 2.47 | 2.74 | |
| 0 | 5.29 | 1.1 | 0.19 | 0.9 | | | | Not compatible |
| 0.5 | 5.54 | 5.3 | 0.07 | 5.1 | 0.12 | | | 50° C./16.7% |
| 1 | 5.83 | | | | | | | LOD |
| 2 | 6.41 | | | | | | | |
| 3 | 8.78 | | | | | | | |
| 6 | 14.78 | | | | | | | |

Granulate J Carmellose Calcium/Atorvastatin 5:1

| Stability 40° C./75% RH | | | Detailed impurities (RRT) | | | |
|---|---|---|---|---|---|---|
| Time (mths) | LOD | Total Imp | Desfl | Lact | 1.15 | |
| 0 | 6.69 | 1.1 | 0.18 | 0.9 | | Not compatible |
| 0.5 | 7.98 | 7.34 | 0.07 | 7.13 | 0.15 | 50° C./16.7% LOD |
| 1 | 8.93 | | | | | |
| 2 | 10.63 | | | | | |
| 3 | 11.95 | | | | | |
| 6 | | | | | | |

The results of Experiment 1 indicate that crystalline atorvastatin calcium form VI is compatible with lactose, pregelatinized starch (tested as starch 1500) and thus probably with conventional starch, CaCO₃, Crospovidone and Aerosil®, even at a high LOD level (such as 16.7%); almost compatible with microcrystalline cellulose (tested in the form of Avicel); but is not compatible with Croscarmellose sodium, Carmellose calcium, sodium starch glycolate and stearic acid.

According to these results, cores of the present invention according to preferred embodiments optionally and preferably comprise crystalline atorvastatin calcium form VI as an active ingredient (although optionally another crystalline form may be used, including but not limited to any polymorph form, such as crystalline form I, II and so forth) and pregelatinized starch such as starch 1500 and/or lactose and/or a combination thereof as major compatible excipients. Such cores may optionally comprise one or more of HPC, HPMC, PVP (binders), Crospovidone (as a disintegrant), Tween® (as a surfactant), magnesium stearate (as a lubricant), Aerosil® (tabletting aid), microcrystalline cellulose such as Avicel) and maybe mannitol although not tested (as fillers) as minor compatible excipients. Without wishing to be limited by a single hypothesis, such cores would probably be stable without the need of stabilizing agent even if these major or minor compatible excipients are wet granulated with the active crystalline atorvastatin calcium form VI.

Preferably, croscarmellose sodium, carmellose calcium, sodium starch glycolate and stearic acid should not be used in the formula. If used, they preferably should be used as extragranular excipient or as very minor intragranular excipients.

Experiment 2: Compatibility of Amorphous Atorvastatin Calcium with Excipients which were Found Compatible with Atorvastatin Ca Crystalline Form VI

After it was shown that crystalline atorvastatin calcium form VI was compatible and stable with certain excipients, experiments were performed to determine the stability of amorphous Atorvastatin calcium when prepared with those excipients.

Details of the experiment: amorphous atorvastatin calcium was mixed with each tested excipient either at the ratio 1:9 or at the ratio 4:6. One gram of each blend was mixed with 200 μl purified water (LOD of the blend was 16.67%), placed in a closed glass vial and placed for 2 weeks in an incubator at 50° C. for a compatibility test.

TABLE 6
Compatibility of amorphous atorvastatin calcium with the different excipients (2
weeks-50° C.-16.7% LOD).

| | Ratio | | | | | Max |
|---|---|---|---|---|---|---|
| | (atorvastatin | LOD at | Total | | | unknown |
| Excipient tested | to excipient) | t = 0 | Impurities | Lactone | Desfluoro | impurity |
| Reference (no | NA | 16.67% | 0.33 | 0.04 | 0.12 | 0.05 |
| excipient | | | | | | |
| added) | | | | | | |
| Starch 1500 | 1:9 | 16.67% | 0.14 | 0.05 | 0.05 | 0.04 |
| Starch 1500 | 4:6 | 16.67% | 0.09 | 0.08 | 0.01 | — |
| Lactose | 1:9 | 16.67% | 0.18 | 0.07 | 0.05 | 0.04 |
| Lactose | 4:6 | 16.67% | 0.05 | 0.02 | 0.01 | 0.02 |
| MCC PH101 | 1:9 | 16.67% | 0.18 | 0.08 | 0.04 | 0.03 |
| MCC PH101 | 4:6 | 16.67% | 0.11 | 0.1 | — | 0.01 |
| MCC PH102 | 1:9 | 16.67% | 0.19 | 0.09 | 0.04 | 0.02 |
| MCC PH102 | 4:6 | 16.67% | 0.09 | 0.09 | — | — |
| CaCO₃ | 1:9 | 16.67% | 0.07 | — | 0.04 | 0.02 |
| CaCO₃ | 4:6 | 16.67% | 0.01 | — | 0.01 | — |
| Stearic Acid | 9:1 | 16.67% | 4.55 | 4.45 | 0.10 | — |
| Aerosil ® | 7:3 | 16.67% | 0.12 | — | 0.12 | — |
| Mannitol | 1:9 | 16.67% | 0.92 | 0.54 | 0.01 | 0.23 |
| Ethanol | 8:2 | 0% | 1.73 | 0.19 | 0.02 | 0.46 |
| Isopropyl | 8:2 | 0% | 2.43 | 0.41 | 0.04 | 1.24 |
| Alcohol | | | | | | |

MCC: Microcrystalline Cellulose

These results show that amorphous a Atorvastatin calcium is also compatible with Lactose, pregelatinized starch (such as Starch 1500, and thus probably with conventional starch), Avicel, CaCO₃ (known in the art as a stabilizer), Crospovidone (although not tested here) and Aerosil( even at a high LOD level (such as 16.7%); almost compatible with mannitol; not compatible with ethanol, isopropyl alcohol, stearic acid, and presumably not compatible with Croscarmellose sodium (not tested), Carmellose calcium (not tested), sodium Starch Glycolate (not tested) at the ratios tested.

Furthermore, these results show that cores that contain amorphous atorvastatin calcium as an active ingredient, one or more of starch, such as pregelatinized starch (such as Starch 1500) and/or lactose and/or optionally microcrystalline cellulose (Avicel) as major compatible excipients; one or more of PC, HPMC, or PVP as binders; Crospovidone (as a disintegrant), Tween® (as a surfactant), Magnesium stearate (lubricant), Aerosil® (tabletting aid), and Mannitol as minor compatible excipients should probably be stable without the need of stabilizing agent even if these major or minor compatible excipients are wet granulated with the active amorphous atorvastatin calcium.

Preferably, croscarmellose sodium, carmellose calcium, sodium starch glycolate and stearic acid should not be used in the formula. If used, they preferably should be used as extragranular excipient or as very minor intragranular excipients.

Also, these results show that it is preferable to use water rather than ethanol or isopropyl alcohol in the granulation process of amorphous atorvastatin calcium with other excipients. In general this is true also for the crystalline forms.

Experiment 3: Stability of Amorphous Atorvastatin Calcium Granulated with Various Excipients

Amorphous atorvastatin calcium proved to be compatible when mixed with certain excipients. It was also important to test it when granulated with the same excipients.

Details of the experiment: 3 g of amorphous atorvastatin calcium and 15 g of the excipient tested were granulated manually with mortar and pestle using between 3 and 5 ml granulation solution containing water, Klucel® LF and Tween® 80. The exact formula of each wet granulate is detailed below:

TABLE 7
Formula of the wet granulates with amorphous atorvastatin calcium

| | Amorphous | | | | | |
|---|---|---|---|---|---|---|
| | Atorvastatin | | Klucel ® | Tween ® | Water | % |
| Granulate | calcium | Excipient | LF (g) | (g) | (g) | LOD |
| K | 3 g | 15 amorphous | 0.288 | 0.058 | 5 | ~20% |
| (reference) | | Atorvastatin | | | | |
| | | calcium | | | | |
| L | 3 g | 15 g lactose | 0.172 | 0.034 | 3 | ~17% |
| M | 3 g | 15 g CaCO₃ | 0.288 | 0.058 | 5 | ~20% |
| N | 3 g | 15 g Avicel ® | 0.288 | 0.058 | 5 | ~20% |
| | | PH101 | | | | |
| O | 3 g | 15 g Avicel ® | 0.288 | 0.058 | 5 | ~20% |
| | | PH102 | | | | |
| P | 3 g | 15 g Crospovidone | 0.288 | 0.058 | 5 | ~20% |
| Q | 3 g | 15 g Starch 1500 | 0.288 | 0.058 | 5 | ~20% |

The wet granulates were placed in an oven at 50° C. for 1 or 2 days for drying. Then the dry granulate were sieved through a 600μ sieve and checked for LOD to be less than 5%.

Each dry granulate was placed in a 34 ml Securitainer® which is a plastic container for containing medicine, and placed in an incubator at 40° C./75% RH for 6 month stability testing.

Results of this stability study are summarized in the following table.

TABLE 8
Stability (40° C./75% RH) of amorphous atorvastatin
calcium granulated with the different excipients tested.

| Granulate | Excipient tested | LOD at t = 0 | Stability |
|---|---|---|---|
| K | Reference w/o | TBD | Stable only 3 months. |
| | excipient | | LOD 3.65% |
| L | Lactose | TBD | Stable only 3 months. |
| | | | LOD 0.59% |
| M | CaCO₃ | TBD | Stable 6 months. |
| | | | LOD 0.69% |
| N | Avicel PH101 | TBD | Stable only 1 months. |
| | | | LOD 2.44% |
| O | Avicel PH102 | TBD | Stable only 2 months. |
| | | | LOD 3.58% |
| P | Crospovidone | TBD | Stable only 3 months. |
| | | | LOD 12.08% |
| Q | Starch 1500 | TBD | Stable only 3 months. |
| | | | LOD 7.08% |

TABLE 9
Stability and compatibility of amorphous atorvastatin calcium with the different
excipients tested (detailed presentation of the results)
Granulate K Reference (Atorvastatin calcium alone)

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl. | Lact. | 0.74 | 1.57 | 2.48 | 2.74 | |
| 0 | NC | 0.14 | 0.14 | | | | | | Stable |
| 1 | +2.41% | 0.06 | 0.06 | | | | | | Stable |
| 2 | +3.41% | 0.5 | 0.17 | | | | 0.24 | 0.09 | Stable |
| 3 | +3.65% | 0.69 | 0.13 | | | | 0.35 | 0.14 | Stable |
| 6 | +3.69% | 1.31 | 0.09 | 0.12 | 0.08 | 0.11 | 0.61 | 0.3 | Unstable |

Granulate L Lactose/Atorvastatin 5:1

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl | Lact | 0.74 | 1.45 | 1.5 | 2.74 | Stable |
| 0 | NC | 0.14 | 0.14 | | | | | | Stable |
| 1 | +0.44% | 0.05 | 0.05 | | | | | | Stable |
| 2 | +0.52% | 0.32 | 0.18 | 0.13 | | | | | Stable |
| 3 | +0.59% | 0.66 | 0.14 | 0.27 | | | | | Stable |
| 6 | +0.50% | 1.58 | 0.09 | 0.67 | 0.07 | 0.09 | 0.36 | 0.3 | Unstable |

Granulate M CaCO₃/Atorvastatin 5:1

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl | Lact | 0.74 | 1.57 | 2.48 | 2.74 | |
| 0 | NC | 0.13 | 0.13 | | | | | | Stable |
| 1 | +0.41% | 0.06 | 0.06 | | | | | | Stable |
| 2 | +0.68% | 0.45 | 0.17 | | | | 0.18 | | Stable |
| 3 | +0.77% | 0.76 | 0.13 | 0.06 | 0.05 | | 0.28 | | Stable |
| 6 | +0.69% | 1.2 | 0.08 | 0.1 | 0.09 | 0.14 | 0.5 | 0.28 | Stable |

Granulate N Avicel PH101/Atorvastatin 5:1

| Stability 40° C./75% RH | | | | | | | | | |
|---|---|---|---|---|---|---|---|---|---|
| Time | | Total | Detailed impurities (RRT) | | | | | | |
| (mths) | LOD | Imp | Desfl | Lact | 0.74 | 1.15 | 2.48 | 2.74 | |
| 0 | NC | 0.24 | 0.14 | 0.1 | | | | | Stable |
| 1 | +2.44% | 0.22 | 0.05 | 0.17 | | | | | Stable |
| 2 | +3.7% | 0.95 | 0.16 | 0.66 | | | | 0.13 | Unstable |
| 3 | +3.89% | 1.56 | 0.1 | 0.93 | 0.05 | 0.11 | 0.14 | 0.23 | Unstable |
| 6 | +4.05% | 2.33 | 0.05 | 1.03 | 0.09 | 0.24 | 0.3 | 0.38 | Unstable |

Granulate O Avicel PH102/Atorvastatin 5:1

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl | Lact | 0.74 | 1.15 | 2.48 | 2.74 | |
| 0 | NC | 0.24 | 0.13 | 0.11 | | | | | Stable |
| 1 | +0.59% | 0.17 | 0.05 | 0.12 | | | | | Stable |
| 2 | +3.58% | 0.78 | 0.16 | 0.5 | | | | 0.13 | Stable |
| 3 | +4.07% | 1.57 | 0.09 | 0.92 | 0.05 | 0.1 | 0.1 | 0.24 | Unstable |
| 6 | +%3.97 | 2.35 | 0.05 | 1.04 | 0.1 | 0.25 | 0.3 | 0.38 | Unstable |

Granulate P Crospovidone/Atorvastatin 5:1

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl | Lacto | 0.74 | 1.45 | 2.48 | 2.74 | |
| 0 | NC | 0.21 | 0.13 | 0.08 | | | | | Stable |
| 1 | +3.78% | 0.05 | 0.05 | | | | | | Stable |
| 2 | +8.90% | 0.42 | 0.14 | | | | 0.17 | 0.11 | Stable |
| 3 | +12.08% | 0.65 | 0.12 | | | | 0.21 | 0.23 | Stable |
| 6 | +15.07% | 1.64 | | 0.32 | 0.13 | 0.06 | 0.53 | 0.55 | Unstable |

Granulate P Starch 1500/Atorvastatin 5:1

| Stability 40 C./75% RH | | | | | | | | | |
|---|---|---|---|---|---|---|---|---|---|
| Time | | Total | Detailed impurities (RRT) | | | | | | |
| (mths) | LOD | Imp | Desfl | Lact | 0.74 | 1.5 | 2.48 | 2.74 | |
| 0 | NC | 0.23 | 0.13 | 0.1 | | | | | Stable |
| 1 | +1.51% | 0.13 | 0.06 | 0.08 | | | | | Stable |
| 2 | +6.47% | 0.65 | 0.16 | 0.28 | | | 0.14 | 0.06 | Stable |
| 3 | +7.08% | 0.98 | 0.12 | 0.48 | | 0.05 | 0.12 | 0.21 | Stable |
| 6 | +7.23% | 1.45 | 0.08 | 0.57 | 0.06 | | 0.34 | 0.35 | Unstable |

Experiment 3 showed that more impurities appear when testing the above granulated material for stability at 40° C./75% RH for a long time than during the 15 day compatibility tests at 50° C. of Experiment 2. The results provided a similar demonstration of compatibility as compared to Experiment 2 and thus the same conclusions except that optionally and preferably microcrystalline cellulose (such as Avicel) should preferably be a minor “almost” compatible excipient in the formula rather than a major one even if the LOD of the formula remained low (<3.5%).

Experiment 4: Production of 150 mg Cores Containing Either 10 mg Amorphous or 10 mg Crystalline Atorvastatin Calcium form VI and Various Ratios of Starch 1500 and Lactose as Major Excipients

Description of the Experiment:

Briefly, Cores #1 to #4 were produced by mixing the blend for granulation before granulating it with the granulation solution containing Tween 80 and water. Typically, the LOD of wet granulates was between 20% and 30%. The wet granulates were dried in oven at 60° C. for several hours to allow the LOD to decrease below 3-5%. The dry granulates were milled through a 0.5 mm sieve before adding the extra-granular excipients and compressing the final blends to round 8 mm diameter cores. The details of the 4 formulations are listed in the following table:

TABLE 10
Core and batch formula

| | Core #1 (30% | | | Core #2 (70% | | | Core #3 (30% | | | Core #4 | | |
|---|---|---|---|---|---|---|---|---|---|---|---|---|
| | Starch 1500 | | | Starch 1500 | | | Starch 1500 | | | (70% Starch 1500 | | |
| | 62% Lactose) | | | 22% Lactose) | | | 62% Lactose) | | | 22% Lactose) | | |
| | Core | Batch | % | Core | Batch | % | Core | Batch | % | Core | Batch | % |
| ← | 10.32 | 8.00 | 6.88 | 10.32 | 8.00 | 6.88 | | | | | | |
| atorvastatin | | | | | | | | | | | | |
| Ca | | | | | | | | | | | | |
| amorphous | | | | | | | | | | | | |
| ← | | | | | | | 10.32 | 8.00 | 6.88 | 10.32 | 8.00 | 6.88 |
| atorvastatin | | | | | | | | | | | | |
| ca form VI | | | | | | | | | | | | |
| ← Starch | 37.5 | 29.07 | 25.00 | 97.5 | 75.58 | 65.00 | 37.5 | 29.07 | 25.00 | 97.5 | 75.58 | 65.00 |
| 1500 | | | | | | | | | | | | |
| ← Lactose | 93.4 | 72.37 | 62.24 | 33.4 | 25.86 | 22.24 | 93.4 | 72.37 | 62.24 | 33.4 | 25.86 | 22.24 |
| monoh. | | | | | | | | | | | | |
| 100M | | | | | | | | | | | | |
| ↑ Tween 80 | 0.57 | 0.44 | 0.38 | 0.57 | 0.44 | 0.38 | 0.57 | 0.44 | 0.38 | 0.57 | 0.44 | 0.38 |
| →Starch | 7.5 | 5.81 | 5.00 | 7.5 | 5.81 | 5.00 | 7.5 | 5.81 | 5.00 | 7.5 | 5.81 | 5.00 |
| 1500 | | | | | | | | | | | | |
| →Mg | 0.75 | 0.58 | 0.50 | 0.75 | 0.58 | 0.50 | 0.75 | 0.58 | 0.50 | 0.75 | 0.58 | 0.50 |
| Stearate | | | | | | | | | | | | |
| Total | 150.0 | 116.3 | 100.0 | 150.0 | 116.3 | 100.0 | 150.0 | 116.3 | 100.0 | 150.0 | 116.3 | 100.0 |

Key to table 10:
← Granulation blend;
↑ Granulation solution
→ Additional extragranular excipients in final blend

Cores #1 to 4 were tested at t=0 for their content, impurities and compared with Lipitor for their dissolution profile.

The dissolution tests were performed in 900 ml intestinal buffer pH 6.8 using paddles at 50 rpm. The concentration of Atorvastatin was determined using a spectrophotometer λmax=248 nm. The optic length of the cell was 1 cm.

Results are summarized in the following tables and figures.

TABLE 11
Properties of cores #1 to 4 containing amorphous or crystalline form VI
Atorvastatin Calcium and Starch 1500 and Lactose at t = 0

| | Content (% | | Dissolution % in | | |
|---|---|---|---|---|---|
| Core # | of LC) | Impurities | IF pH 6.8 | | Remarks |
| Core #1 | 91% | Max known | 0 min | 0 | Reasons for |
| | | Not detected | 5 min | 77.2 | low content are |
| | | Max unknown | 15 min | 103.6 | currently |
| | | <0.05% | 30 min | 108.1 | checked |
| | | Total | 60 min | 107.3 | |
| | | <0.05% | | | |
| Core #2 | 101.2% | Max known | 0 min | 0 | |
| | | Not detected | 5 min | 54 | |
| | | Max unknown | 15 min | 93.8 | |
| | | <0.05% | 30 min | 101 | |
| | | Total | 60 min | 102 | |
| | | <0.05% | | | |
| Core #3 | 90.4% | Max known | 0 min | 0 | Reasons for |
| | | Not detected | 5 min | 47.1 | low content are |
| | | Max unknown | 15 min | 62.7 | currently |
| | | <0.05% | 30 min | 67.7 | checked |
| | | Total | 60 min | 78.5 | |
| | | <0.05% | 120 min | 97.5 | |
| Core #4 | 98.7% | Max known | 0 min | 0 | |
| | | Not detected | 5 min | 77.7 | |
| | | Max unknown | 15 min | 104.0 | |
| | | <0.05% | 30 min | 106.4 | |
| | | Total | 60 min | 109 | |
| | | <0.05% | | | |
| Ref Lipitor | ~100% | “Stable” for at | 0 min | 0 | |
| 20 mg tablet | | least 6 months at | 5 min | 70.5 | |
| | | 40 C and 12 | 15 min | 83.7 | |
| | | months at 30 C | 30 min | 89.3 | |
| | | | 60 min | 92.0 | |

These results show that the preferred embodiments of formulations according to the present invention are compatible and are stable even when using the less stable amorphous form of Atorvastatin.

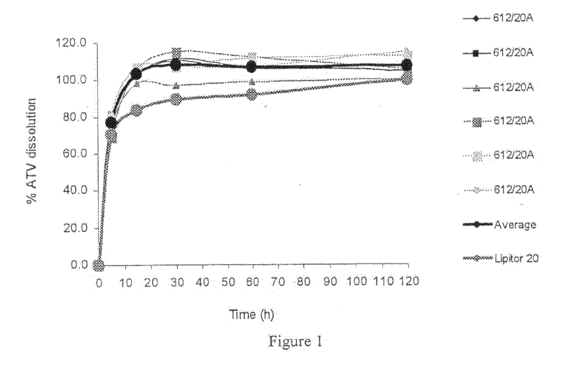

FIGS. 1 to 4 show that core formulations 1-4 are able to provide dissolution profiles as fast as innovator's Lipitor tablet (20 mg Atorvastatin formulation used).

FIG. 1 shows the dissolution release profile in IF (intestinal fluid) pH 6.8 for the Amorphous Atorvastatin Calcium core #1 containing 30% starch 1500 and 62% lactose monohydrate (uncoated). The amount of amorphous atorvastatin base is 10 mg per tablet.

TABLE 12
Raw data (ppm)

| Time (min) | Core #1 | Core #1 | Core #1 | Core #1 | Core #1 | Core #1 |
|---|---|---|---|---|---|---|
| 0 | 0 | 0 | 0 | 0 | 0 | 0 |
| 5 | 8.55 | 9.04 | 8.28 | 7.69 | 8.82 | 9.05 |
| 15 | 11.36 | 11.42 | 10.91 | 11.67 | 11.91 | 11.91 |
| 30 | 12.27 | 12.4 | 10.81 | 12.84 | 11.95 | 11.93 |
| 60 | 11.81 | 11.91 | 11.01 | 12.52 | 12.47 | 11.94 |
| 120 | 12.04 | 11.67 | 11.13 | 11.73 | 12.56 | 12.83 |

TABLE 13
Percent release of atorvastatin

| Time | | | | | | | | | |
|---|---|---|---|---|---|---|---|---|---|
| (min) | Core #1 | Core #1 | Core #1 | Core #1 | Core #1 | Core #1 | Average | SD | Lipitor 20 |
| 0 | 0.0 | 0.0 | 0.0 | 0.0 | 0.0 | 0.0 | 0.0 | — | 0.0 |
| 5 | 77.0 | 81.4 | 74.5 | 69.2 | 79.4 | 81.5 | 77.2 | 6.1 | 70.5 |
| 15 | 102.1 | 102.6 | 98.0 | 104.8 | 107.0 | 107.0 | 103.6 | 3.3 | 83.7 |
| 30 | 110.2 | 111.3 | 97.2 | 115.1 | 107.4 | 107.2 | 108.1 | 5.6 | 89.3 |
| 60 | 106.1 | 107.0 | 98.9 | 112.3 | 112.0 | 107.3 | 107.3 | 4.6 | 92.0 |
| 120 | 108.0 | 104.9 | 100.0 | 105.4 | 112.8 | 115.1 | 107.7 | 5.1 | 100.3 |

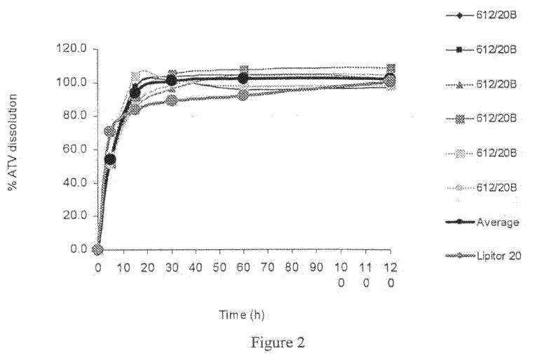

FIG. 2 shows the dissolution release profile in IF (intestinal fluid) pH 6.8 for the amorphous atorvastatin calcium core #2 containing 70% starch 1500 and 22% lactose monohydrate. Core #2 comprises amorphous atorvastatin calcium (10 mg of base).

TABLE 14
Raw data (ppm)

| Time (min) | Core #2 | Core #2 | Core #2 | Core #2 | Core #2 | Core #2 |
|---|---|---|---|---|---|---|
| 0 | 0 | 0 | 0 | 0 | 0 | 0 |
| 5 | 6.25 | 5.91 | 6.25 | 5.76 | 6.25 | 5.53 |
| 15 | 10.92 | 10.88 | 9.46 | 10.2 | 11.57 | 9.68 |
| 30 | 11.5 | 11.34 | 10.72 | 11.75 | 11.3 | 10.98 |
| 60 | 11.66 | 10.66 | 11.66 | 11.99 | 10.88 | 11.52 |
| 120 | 11.66 | 10.76 | 11.47 | 12.13 | 10.88 | 11.66 |

TABLE 15
Percent release of Atorvastatin

| Time | | | | | | | | | |
|---|---|---|---|---|---|---|---|---|---|
| (min) | Core #2 | Core #2 | Core #2 | Core #2 | Core #2 | Core #2 | Average | SD | Lipitor 20 |
| 0 | 0.0 | 0.0 | 0.0 | 0.0 | 0.0 | 0.0 | 0.0 | — | 0.0 |
| 5 | 56.3 | 53.3 | 56.3 | 51.8 | 56.3 | 49.8 | 54.0 | 5.2 | 70.5 |
| 15 | 98.0 | 97.6 | 84.9 | 91.6 | 103.8 | 86.9 | 93.8 | 7.7 | 83.7 |
| 30 | 103.1 | 101.7 | 96.1 | 105.3 | 101.4 | 98.4 | 101.0 | 3.3 | 89.3 |
| 60 | 104.6 | 95.7 | 104.4 | 107.4 | 97.7 | 103.1 | 102.2 | 4.4 | 92.0 |
| 120 | 104.6 | 96.6 | 102.7 | 108.6 | 97.8 | 104.4 | 102.5 | 4.4 | 100.3 |

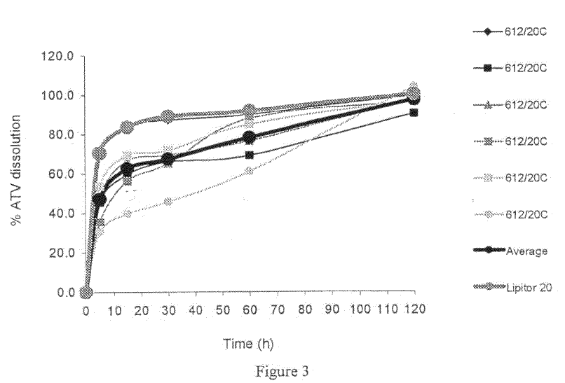

FIG. 3 shows the dissolution release profile in IF (intestinal fluid) pH 6.8 for the Crystalline form VI Atorvastatin Calcium Core #3, comprising 30% Starch 1500 and 62% lactose monohydrate. Core #3 comprises amorphous atorvastatin calcium (10 mg of base).

TABLE 16
Raw data (ppm)

| Time (min) | Core #3 | Core #3 | Core #3 | Core #3 | Core #3 | Core #3 |
|---|---|---|---|---|---|---|
| 0 | 0 | 0 | 0 | 0 | 0 | 0 |
| 5 | 7.72 | 5.06 | 5.33 | 3.96 | 5.96 | 3.37 |
| 15 | 9.36 | 6.63 | 7.42 | 6.29 | 7.72 | 4.42 |
| 30 | 9.72 | 7.37 | 7.75 | 7.27 | 8 | 5.12 |
| 60 | 10.05 | 7.74 | 8.57 | 9.88 | 9.5 | 6.85 |
| 120 | 11.14 | 10.15 | 10.94 | 10.84 | 10.85 | 11.77 |

TABLE 17
Percent of release of atorvastatin

| Time | | | | | | | | | |
|---|---|---|---|---|---|---|---|---|---|
| (min) | Core #3 | Core #3 | Core #3 | Core #3 | Core #3 | Core #3 | Average | SD | Lipitor 20 |
| 0 | 0.0 | 0.0 | 0.0 | 0.0 | 0.0 | 0.0 | 0.0 | — | 0.0 |
| 5 | 69.5 | 45.5 | 48.0 | 35.7 | 53.7 | 30.4 | 47.1 | 29.4 | 70.5 |
| 15 | 84.2 | 59.6 | 66.7 | 56.5 | 69.4 | 39.7 | 62.7 | 23.7 | 83.7 |
| 30 | 87.4 | 66.2 | 69.6 | 65.2 | 71.9 | 45.9 | 67.7 | 19.7 | 89.3 |
| 60 | 90.2 | 69.4 | 76.8 | 88.1 | 85.1 | 61.2 | 78.5 | 14.6 | 92.0 |
| 120 | 99.7 | 90.4 | 97.4 | 96.5 | 96.9 | 104.1 | 97.5 | 4.6 | 100.3 |

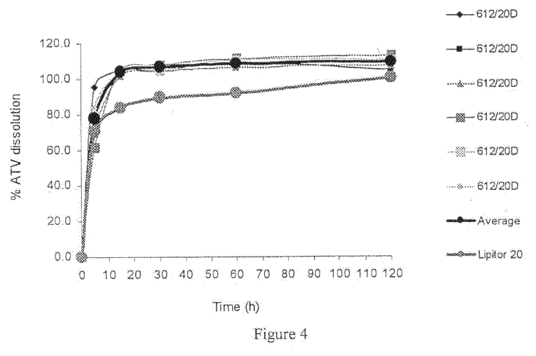

FIG. 4 shows the dissolution release profile in IF (intestinal fluid) pH 6.8 for the Crystalline form VI Atorvastatin Calcium core #4, comprising 70% Starch 1500 and 22% lactose monohydrate. Core #4 comprises amorphous atorvastatin calcium (10 mg of base).

TABLE 18
Raw data (ppm)

| Time | | | | | | |
|---|---|---|---|---|---|---|
| (min) | Core #4 | Core #4 | Core #4 | Core #4 | Core #4 | Core #4 |
| 0 | 0 | 0 | 0 | 0 | 0 | 0 |
| 5 | 10.6 | 8.62 | 7.7 | 6.83 | 8.85 | 9.22 |
| 15 | 11.65 | 11.54 | 11.31 | 11.65 | 11.65 | 11.64 |
| 30 | 12.02 | 11.65 | 11.65 | 12.03 | 11.71 | 11.97 |
| 60 | 11.98 | 12.21 | 11.87 | 12.46 | 12.37 | 11.96 |
| 120 | 12.31 | 11.65 | 11.96 | 12.55 | 12.32 | 12.38 |

TABLE 19
Percent of release of Atorvastatin

| Time | | | | | | | | | |
|---|---|---|---|---|---|---|---|---|---|
| (min) | Core #4 | Core #4 | Core #4 | Core #4 | Core #4 | Core #4 | Average | SD | Lipitor 20 |
| 0 | 0.0 | 0.0 | 0.0 | 0.0 | 0.0 | 0.0 | 0.0 | — | 0.0 |
| 5 | 95.4 | 77.6 | 69.3 | 61.5 | 79.7 | 83.0 | 77.8 | 15.0 | 70.5 |
| 15 | 104.8 | 103.7 | 101.6 | 104.6 | 104.7 | 104.6 | 104.0 | 1.2 | 83.7 |
| 30 | 108.1 | 104.7 | 104.6 | 107.9 | 105.2 | 107.6 | 106.4 | 1.6 | 89.3 |
| 60 | 107.7 | 109.6 | 106.5 | 111.7 | 111.0 | 107.5 | 109.0 | 1.9 | 92.0 |
| 120 | 110.6 | 104.7 | 107.3 | 112.5 | 110.6 | 111.1 | 109.5 | 2.6 | 100.3 |

Cores #1 to #4 were placed in Securitainer® plastic containers, placed for 6 to 12 months in stability at 40 C/75% RH and 30 C/65% RH and regularly tested for weight, assay, and impurities.

Results of these stability tests are summarized in the following tables.

TABLE 20
Core #1

| | | Total | | | RRT | RRT | |
|---|---|---|---|---|---|---|---|
| | LOD | Imp | Des | Lactone | 2.42 | 2.74 | Stability |
| T = 0 | 3.3% | 0.15 | 0.15 | | | | — |
| 30 C./6 MO | 4.2% | 1.06 | 0.11 | 0.17 | 0.37 | 0.41 | Stable |
| 30 C./9 MO | 5.16% | 1.52 | 0.13 | 0.31 | 0.49 | 0.26 | Unstable |
| 30 C./12 MO | 4.78% | 1.72 | 0.13 | 0.25 | 0.81 | 0.35 | Unstable |
| 40 C./3 MO | 5.60% | 1.1 | 0.11 | 0.35 | 0.27 | 0.34 | Stable |
| 40 C./6 MO | 6.01% | 2.05 | 0.08 | 0.43 | 0.59 | 0.58 | Unstable |

TABLE 21
Core #2

| | | Total | | | RRT | RRT | |
|---|---|---|---|---|---|---|---|
| | LOD | Imp | Des | Lactone | 2.42 | 2.74 | Stability |
| T = 0 | 5.39% | 0.27 | 0.17 | 0.1 | | | — |
| 30 C./3 MO | 6.94% | 0.71 | 0.12 | 0.22 | 0.13 | 0.23 | Stable |
| 30 C./6 MO | 7.34% | 1.6 | 0.13 | 0.34 | 0.41 | 0.54 | Unstable |
| 30 C./9 MO | 9.78% | 2.04 | 0.14 | 0.57 | 0.56 | 0.34 | Unstable |
| 40 C./2 MO | 10.58% | 0.31 | 0.05 | 0.26 | | | Stable |
| 40 C./3 MO | 11.91% | 1.5 | 0.1 | 0.74 | | | Unstable |
| 40 C./6 MO | 11.78% | 2.74 | 0.08 | 0.86 | 0.57 | 0.69 | Unstable |

TABLE 22
Core #3

| | | Total | | | RRT | RRT | |
|---|---|---|---|---|---|---|---|
| | LOD | Imp | Des | Lactone | 2.42 | 2.74 | Stability |
| T = 0 | 3.7% | 0.12 | 0.12 | | | | — |
| 30 C./3 MO | 4.56% | 0.39 | 0.09 | 0.08 | 0.07 | 0.15 | Stable |
| 30 C./6 MO | 4.45% | 0.68 | 0.09 | 0.08 | 0.19 | 0.28 | Stable |
| 30 C./9 MO | 5.48% | 0.87 | 0.10 | 0.15 | 0.30 | 0.20 | Stable |
| 30 C./12 MO | 5.17% | 0.94 | 0.09 | 0.19 | 0.45 | 0.21 | Stable |
| 40 C./3 MO | 0.71% | 0.71 | 0.08 | 0.29 | 0.14 | 0.20 | Stable |
| 40 C./6 MO | 6.22% | 1.02 | 0.06 | 0.22 | 0.25 | 0.36 | Stable |

TABLE 23
Core #4

| | | Total | | | | | |
|---|---|---|---|---|---|---|---|
| | LOD | Imp | Des | Lactone | 2.42 | 2.74 | Stability |
| T = 0 | 5.22% | 0.17 | 0.12 | 0.05 | | | — |
| 30 C./3 MO | 6.82% | 0.54 | 0.09 | 0.15 | 0.10 | 0.19 | Stable |
| 30 C./6 MO | 7.41% | 0.92 | 0.09 | 0.23 | 0.23 | 0.37 | Stable |
| 30 C./9 MO | 9.58% | 0.88 | 0.11 | 0.31 | 0.20 | 0.21 | Stable |
| 30 C./12 MO | 8.86% | 1.36 | 0.10 | 0.38 | 0.55 | 0.28 | Unstable |
| 40 C./3 MO | 10.67% | 0.98 | 0.08 | 0.47 | 0.16 | 0.26 | Stable |
| 40 C./6 MO | 11.41% | 1.59 | 0.06 | 0.49 | 0.30 | 0.46 | Unstable |

The results of Experiment 4 show that preferred embodiments of the formulation according to the present invention could be compressed to tablets which were able to display in most of the cases a dissolution rate that was as fast or even faster than Lipitor®, independently whether amorphous of crystalline form VI Atorvastatin Calcium was used. This means that such tablets would presumably be bioequivalent to Lipitor (the product of the originator).

When crystalline form VI Atorvastatin Calcium was used, preferred embodiments of the formulation according to the present invention compressed to tablets was rather stable both at 30 C and 40 C although these tablets were stored in simple plastic bottles and their LOD was very significantly increased to be above 3.5%.

When amorphous Atorvastatin Calcium was used, preferred embodiments of the formulation according to the present invention compressed to tablets were moderately stable after 6 months at 30 C and 3 months 40° C., while the LOD of the tablets also very significantly increased to be above 3.5%. Any potential storage problems for such formulations therefore could presumably be solved by use of better packaging materials such as Alu/Alu blisters which would protect them from air and humidity.

### Experiment 5: Production of 300 mg Cores Containing Either 20 mg Amorphous or 20 mg Crystalline Atorvastatin Calcium Form VI and Various Ratios of Starch 1500 and Lactose as Major Excipients

Similar formulations were used as for cores #1 to #4 from experiment 4; however the production methods used pilot equipment, for example by performing the granulation and drying steps in a high shear granulator and fluidized bed dryer, or both steps in a low shear V-cone granulator, and compressing the cores in a production scale regular tablet press. The tablets were coated with a standard Opadry II® coating and then packaged in Alu/Alu blisters in order to check the suitability of the formula to standard large scale tablet production processes. This process was also expected to improve the stability of the formula by decreasing the wet granulate drying time, protecting the tablets from oxidation and humidity with the Opadry II® coat and the Alu/Alu packaging.

Description of the Experiment:

Briefly, cores #5, 6, 7, 8, 9 and 10 (whose formulations were rather similar to previous cores #1 to 4 of Experiment 4) were produced by granulating the dry blend with the aqueous granulation solution in a pilot scale Diosna high shear granulator or in a pilot scale V processor low shear granulator. Granulates produced in the high shear granulator were then dried for 48 hours at 60° C. in oven while granulates produced in the V processor were dried in the V processor itself at 60° C. for about 3 hours. The dry granulates were optionally milled if necessary through a 800μ sieve and mixed with the additional extra-granular excipients in an automatic powder blender according to the common state of the art. Typically, the LOD of wet granulates was between 20% and 30% and the LOD of the dry granulate was below 5.5%.

The resulting blends were compressed to 300 mg capsule shapes 13 mm*6 mm cores in a pilot scale 15 station Kilian RLS 15 tablet press.

The resulting cores were then optionally coated with 6 mg to 10 mg Opadry II® coat in a pilot scale “Accelacota” coating pan, according to the common state of the art.

Cores or coated tablets were packaged in Alu/Alu blisters to protect them from light, air, humidity and oxidation. No problems were found in the production process of these tablets.

The detailed formulas of cores #5 to 10 as well as main data about their production process are detailed in Table 24.

Alu Alu Blisters of cores #5 to 10 were stored at 25° C., 30 C/65% RH and 40 C/75% RH. The results of these stability studies are summarized in Tables 25 to 29.

TABLE 24
Core Composition and Percent formula + Process

| | | | | | | | | | | | Core #10 | |
|---|---|---|---|---|---|---|---|---|---|---|---|---|
| | | | | | | | | | | | (20% Starch | |
| | Core #5 | | | | Core #7 | | Core #8 | | Core #9 | | 1500 | |
| | (70% Starch | | | | (70% Starch | | (20% Starch | | (20% Starch | | 70% | |
| | 1500 | | Core #6 (70% | | 1500 | | 1500 | | 1500 | | Lactose + | |
| | 22% | | Starch 1500 | | 22% | | 70% | | 70% Lactose + | | 20% | |
| | Lactose) | | 22% Lactose) | | Lactose) | | Lactose) | | 5% Crospovidone) | | CaCO₃) | |
| | Form VI | | Amorphous | | Form VI | | Amorphous | | Amorphous | | Amorphous | |
| | Mg/ | | Mg/ | | Mg/ | | Mg/ | | Mg/ | | Mg/ | |
| | Core | % | Core | % | Core | % | Core | % | Core | % | Core | % |
| ←Atorvastatin | | | 20.7 | 6.88 | | | 20.7 | 6.9 | 20.7 | 6.9 | 20.7 | 6.88 |
| Ca | | | | | | | | | | | | |
| Amorphous | | | | | | | | | | | | |
| ←Atorvastatin | 20.7 | 6.88 | | | 20.7 | 6.85 | | | | | | |
| Ca form VI | | | | | | | | | | | | |
| ←Starch | 195.3 | 65.0 | 195.3 | 65.0 | 189.5 | 62.8 | 44.4 | 14.8 | 44.4 | 14.8 | 44.4 | 25.00 |
| 1500 | | | | | | | | | | | | |
| ←Lactose | 66.8 | 22.2 | 66.8 | 22.2 | 66.8 | 22.1 | 210 | 70 | 210 | 70 | 210 | 62.24 |
| monoh. 100M | | | | | | | | | | | | |
| ↑Tween ® 80 | 1.14 | 0.38 | 1.14 | 0.38 | 1.14 | 0.38 | 1.2 | 0.4 | 1.2 | 0.4 | 1.2 | 0.38 |
| ↑Klucel ® LF | | | | | 5.81 | 1.92 | 5.7 | 1.9 | 5.7 | 1.9 | 5.7 | |
| →Starch | 15.0 | 5.0 | 15.0 | 5.0 | 15.0 | 4.97 | 15 | 5.0 | 15 | 5.0 | 15 | 5.00 |
| 1500 | | | | | | | | | | | | |
| →Aerosil ® | | | | | 1.50 | 0.5 | 1.5 | 0.5 | 1.5 | 0.5 | 1.5 | 0.5 |
| →Crospovidone | | | | | | | | | 15.0 | 5 | | |
| →CaCO₃ | | | | | | | | | | | 60.0 | 20 |
| →Mg Stear | 1.5 | 0.5 | 1.5 | 0.5 | 1.50 | 0.5 | 1.5 | 0.5 | 1.5 | 0.5 | 1.5 | 0.50 |
| Total core | 300 | 100% | 300.4 | 100% | 302.0 | 100% | 300 | 100% | 300 | 105% | 300 | 120.0% |

| | | | | | | |
|---|---|---|---|---|---|---|
| Opadry ® II | — | — | — | +2% weight | +2% weight | +2% weight |
| coat | | | | increase | increase | increase |
| LOD of final | 5.20% | 5.21% | 5.69% | 4.80% | 4.01% | 3.91% |
| core | | | | | | |
| Disintegration | <3.5 min | <4 min | <5.5 min | <8 min | <5 min | <3 min |
| time in HCl | | | | | | |
| 0.1N⁽*⁾ | | | | | | |
| Disintegration | <3 min | <3 min | <4.5 min | <8.5 min | <5.5 min | <3 min |
| time at pH | | | | | | |
| 6.8⁽**⁾ | | | | | | |

Process

| | High/Low shear | |
|---|---|---|
| | granulation | |
| | High shear Diosna | Low shear V processor |
| Drying | Oven 60° C. 48H | 60° C. in V processor 3H |
| High speed tablet | ✓ | ✓ |
| Press | | |
| Opadry II ® | — | ✓ |
| coating | | |
| Alu/Alu | ✓ | ✓ |
| blistering | | |
| Batch size | 1.5 kg | 5 kg |

Key to table 24:
← Granulation blend;
↑ Granulation solution;
→ Additional extragranular excipients in final blend;
⁽*⁾ & ⁽**⁾<2 min for Lipitor ®

TABLE 25
Stability of Core #5 (70% Starch 1500 22% Lactose) - Form VI

| | | | Dissolution | | | | | |
|---|---|---|---|---|---|---|---|---|
| | Average | | (pH 6.8) | Total | | | RRT | RRT |
| Time | weight | Assay | 5′/15′/30′/45′/60′ | Imp | Desfl. | Lact | 2.42 | 2.74 |

Stability at 25° C.

| | | | | | | | | |
|---|---|---|---|---|---|---|---|---|
| 0 | 307.0 | 18.0 | 67/83/85/87/92 | 0.20 | 0.12 | 0.08 | | |
| 3 | 305.0 | 18.4 | 65/82/86/87/94 | 0.63 | 0.19 | 0.14 | 0.07 | 0.18 |
| 6 | 305.0 | 18.3 | 64/80/83/84/89 | 0.62 | 0.11 | 0.15 | 0.11 | 0.20 |
| 9 | 305.0 | 18.7 | 69/83/88/93/95 | 0.45 | 0.12 | 0.09 | 0.08 | 0.17 |

Stability at 30° C./65% RH

| | | | | | | | | |
|---|---|---|---|---|---|---|---|---|
| 0 | 307.0 | 18.0 | 67/83/85/87/92 | 0.20 | 0.12 | 0.08 | | |
| 3 | 306.0 | 18.9 | 65/83/86/88/93 | 0.59 | 0.13 | 0.14 | 0.08 | 0.19 |
| 6 | 305.0 | 18.0 | 62/78/80/82/88 | 0.69 | 0.11 | 0.16 | 0.16 | 0.20 |
| 9 | 304.0 | 18.6 | 67/83/89/93/94 | 0.58 | 0.11 | 0.08 | 0.22 | 0.17 |

Stability at 40° C./75% RH

| | | | | | | | | |
|---|---|---|---|---|---|---|---|---|
| 0 | 307.0 | 18.0 | 67/83/85/87/92 | 0.20 | 0.12 | 0.08 | | |
| 1 | 305.0 | 18.3 | 67/88/92/93 | 0.10 | 0.10 | 0.12 | | |
| 2 | 305.0 | 18.1 | 68/85/88/90/97 | 0.44 | 0.10 | 0.12 | 0.09 | 0.14 |
| 3 | 305.0 | 18.4 | 58/79/83/85/91 | 0.67 | 0.12 | 0.16 | 0.10 | 0.22 |
| 6 | 303.0 | 17.9 | 61/78/79/84/84 | 0.76 | 0.10 | 0.18 | 0.26 | 0.22 |
| | | Lipitor | 71/84/89/91/92 | | | | | |

TABLE 26
Stability of Core #6 (70% Starch 1500, 22% Lactose) Amorphous

| | | | Dissolution | | | | | |
|---|---|---|---|---|---|---|---|---|
| | Average | | (pH 6.8) | Total | | | RRT | RRT |
| Time | weight | Assay | 5′/15′/30′/45′/60′ | Imp | Desfl. | Lact | 2.42 | 2.74 |

Stability at 25° C.

| | | | | | | | | |
|---|---|---|---|---|---|---|---|---|
| 0 | 306.0 | 18.0 | 45/75/83/90/99 | 0.42 | 0.16 | 0.26 | | |
| 3 | 306.0 | 19.2 | 46/76/83/88/95 | 1.17 | 0.17 | 0.48 | 0.12 | 0.32 |
| 6 | 306.0 | 18.4 | 44/75/82/87/92 | 1.25 | 0.15 | 0.47 | 0.23 | 0.34 |

Stability at 30° C./65% RH

| | | | | | | | | |
|---|---|---|---|---|---|---|---|---|
| 0 | 306.0 | 18.0 | 45/75/83/90/99 | 0.42 | 0.16 | 0.26 | | |
| 2 | 307.0 | | 50/82/90/98/103 | 0.79 | 0.13 | 0.37 | 0.08 | 0.23 |
| 3 | 306.0 | 19.2 | 49/79/85/93/96 | 1.15 | 0.16 | 0.49 | 0.15 | 0.28 |
| 6 | 305.0 | 18.5 | 44/74/80/86/92 | 1.35 | 0.14 | 0.49 | 0.29 | 0.37 |

Stability at 40° C./75% RH

| | | | | | | | | |
|---|---|---|---|---|---|---|---|---|
| 0 | 306.0 | 18.0 | 45/75/83/90/99 | 0.42 | 0.16 | 0.26 | | |
| 1 | 306.0 | 18.5 | 45/76/83/89/97 | 0.42 | 0.16 | 0.26 | | |
| 2 | 306.0 | 17.7 | 53/83/92/95/101 | 1.20 | 0.14 | 0.45 | 0.21 | 0.28 |
| 6 | 306.0 | 17.6 | 45/75/80/83/89 | 1.58 | 0.14 | 0.53 | 0.42 | 0.43 |
| | | Lipitor | 71/84/89/91/92 | | | | | |

TABLE 27
Stability of Core #7 (70% Starch 1500, 22% Lactose) Form VI
Stability at 25° C.

| | Aver | | | Total | | | RRT | RRT |
|---|---|---|---|---|---|---|---|---|
| Time | weight | Assay | Dissolution | Imp | Desfl. | Lact | 2.42 | 2.74 |
| 0 | 312.0 | 18.8 | 53/88/97/97 | 0.50 | 0.11 | 0.20 | 0.06 | 0.14 |
| 3 | 309.0 | 18.4 | 49/86/93/94/100 | 0.58 | 0.11 | 0.17 | 0.12 | 0.19 |
| 6 | 308.0 | 19.4 | 59/90/96/97/98 | 0.72 | 0.11 | 0.20 | 0.21 | 0.20 |

Stability at 30° C./65% RH

| | Average | | | Total | | | | |
|---|---|---|---|---|---|---|---|---|
| Time | weight | Assay | Dissolution | Imp | Desfl. | Lact | 2.42 | 2.74 |
| 0 | 312.0 | 18.8 | 53/88/97/97 | 0.50 | 0.11 | 0.20 | 0.06 | 0.14 |
| 1 | | | | | | | | |
| 2 | 309.0 | 17.9 | 48/86/90/92/94 | 0.69 | <0.05 | 0.25 | 0.22 | 0.18 |
| 3 | 309.0 | 18.4 | 53/91/94/97/100 | 0.61 | 0.11 | 0.21 | 0.13 | 0.18 |
| 6 | 309.0 | 18.5 | 62/88/93/94/97 | 0.80 | 0.10 | 0.24 | 0.26 | 0.20 |

Stability at 40 C./75% RH

| | Average | | | Total | | | | |
|---|---|---|---|---|---|---|---|---|
| Time | weight | Assay | Dissolution | Imp | Desfl. | Lact. | 2.42 | 2.74 |
| 0 | 312.0 | 18.8 | 53/88/97/97 | 0.50 | 0.11 | 0.20 | 0.06 | 0.14 |
| 1 | 308.0 | 18.1 | 53/88/89/90/93 | 0.75 | 0.10 | 0.24 | 0.19 | 0.21 |
| 2 | | | | | | | | |
| 3 | 309.0 | 18.5 | 53/85/88/90/98 | 0.90 | 0.09 | 0.30 | 0.30 | 0.21 |
| 6 | 309.0 | 18.4 | 64/91/98/99/99 | 1.23 | 0.09 | 0.49 | 0.41 | 0.23 |
| | | Lipitor | 71/84/89/91/92 | | | | | |

TABLE 28
Stability of core #8 (20% Starch 1500, 70% Lactose) Amorphous

| | Dissolution | | | | | | | | |
|---|---|---|---|---|---|---|---|---|---|
| | (pH 4.5 + 1% Tween) | Aver | | | Total | | | RRT | RRT |
| Time | 5′/15′/30′/45′/60′ | weight | Hardness | Assay | Imp | Desfl. | Lact. | 2.42 | 2.74 |

Stability at 25° C.

| | | | | | | | | | |
|---|---|---|---|---|---|---|---|---|---|
| 0 | 31/85/87/84/85 | 313.0 | 69.0 | 18.4 | 0.63 | <0.05 | 0.14 | 0.12 | 0.18 |
| 3 | 19/78/94/96/7 | 314.0 | 69.0 | 19.8 | 0.80 | <0.05 | 0.18 | 0.25 | 0.16 |

Stability at 30° C./65% RH

| | | | | | | | | | |
|---|---|---|---|---|---|---|---|---|---|
| 0 | 31/85/87/84/85 | 313.0 | 69.0 | 18.4 | 0.63 | <0.05 | 0.14 | 0.12 | 0.18 |
| 1 | | | | | | | | | |
| 2 | 18/73/86/88/87 | 313.0 | 65.0 | 19.4 | 0.95 | <0.05 | 0.41 | 0.15 | 0.20 |
| 3 | 15/76/95/98/98 | 309.0 | 64.0 | 19.6 | 0.89 | | 0.23 | 0.28 | 0.17 |

Stability at 40° C./75% RH

| | | | | | | | | | |
|---|---|---|---|---|---|---|---|---|---|
| 0 | 31/85/87/84/85 | 313.0 | 69.0 | 18.4 | 0.63 | <0.05 | 0.14 | 0.12 | 0.18 |
| 1 | 13/75/91/93/94 | 314.0 | 68.0 | 19.3 | 0.71 | <0.05 | 0.25 | 0.11 | 0.19 |
| 2 | 22/75/88/89/89 | 314.0 | 64.0 | 19.5 | 0.68 | <0.05 | 0.20 | 0.11 | 0.19 |
| 3 | 27/81/93/94/94 | 313.0 | | 19.3 | 1.30 | | 0.52 | 0.41 | 0.16 |
| Lipitor | 65/82/84/85/84 | | | | | | | | |

TABLE 29
Stability of core #9 (20% Starch 1500, 70% Lactose + 5% crospovidone)
Amorphous
Stability at 25° C.

| | Dissolution | | | | | | | | |
|---|---|---|---|---|---|---|---|---|---|
| | (pH 4.5 + 1% Tween) | Aver. | | | Total | | | RRT | RRT |
| Time | 5′/15′/30′/45′/60′ | weight | Hardness | Assay | Imp | Desfl. | Lact | 2.42 | 2.74 |
| 0 | 31/74/83/83/84 | 328.0 | 71.0 | 18.4 | 0.64 | <0.05 | 0.14 | 0.13 | 0.19 |
| 3 | 35/83/95/97/97 | 311.0 | 71.0 | 18.6 | 0.68 | <0.05 | 0.16 | 0.19 | 0.15 |

Stability at 30° C./65% RH

| | Dissolution | | | | | | | | |
|---|---|---|---|---|---|---|---|---|---|
| | (pH 4.5 + 1% Tween) | Aver. | | | Total | | | RRT | |
| Time | 5′/15′/30′/45′/60′ | weight | Hardness | Assay | Imp | Desfl. | Lact | 2.42 | 2.74 |
| 0 | 31/74/83/83/84 | 328.0 | 71.0 | 18.4 | 0.64 | <0.05 | 0.14 | 0.13 | 0.19 |
| 2 | | 329.0 | 69.0 | 19.6 | 0.74 | <0.05 | 0.21 | 0.11 | 0.20 |
| 3 | 44/83/94/97/98 | 328.0 | | 19.7 | 0.97 | | 0.24 | 0.31 | 0.20 |

Stability at 40° C./75% RH

| | Dissolution | | | | | | | | |
|---|---|---|---|---|---|---|---|---|---|
| | (pH 4.5 + 1% Tween) | | | | | | | | |
| | 5′/15′/30′/45′/ | Aver. | | | Total | | | RRT | |
| Time | 60′ | weight | Hardness | Assay | Imp | Desfl. | Lact | 2.42 | 2.74 |
| 0 | 31/74/83/83/84 | 328.0 | 71.0 | 18.4 | 0.64 | <0.05 | 0.14 | 0.13 | 0.19 |
| 1 | 35/79/89/90/90 | 329.0 | 66.0 | 19.0 | 0.47 | <0.05 | 0.09 | 0.08 | 0.15 |
| 2 | 44/79/88/89/89 | 328.0 | 62.0 | 19.5 | 1.00 | <0.06 | 0.38 | 0.15 | 0.26 |
| 3 | 48/87/95/96/95 | 327.0 | 62.0 | 19.3 | 1.29 | — | 0.46 | 0.45 | 0.18 |

Lipitor 65/82/84/85/84

TABLE 30
Stability of Core #10 (20% Starch 1500, 70% Lactose + 20% Ca CO₃)
Amorphous

| | Dissolution | | | | | | | | |
|---|---|---|---|---|---|---|---|---|---|
| | (pH 4.5 + 1% Tween) | Average | | | Total | | | RRT | RRT |
| Time | 5′/15′/30′/45′/60′ | weight | Hardness | Assay | Imp | Desfl. | Lact | 2.42 | 2.74 |

Stability at 25° C.

| | | | | | | | | | |
|---|---|---|---|---|---|---|---|---|---|
| 0 | 36/60/64/68/73 | 392.0 | 61.0 | 19.8 | 0.66 | <0.05 | 0.17 | 0.07 | 0.24 |
| 3 | 46/73/81/83/89 | 391.0 | 65.0 | 19.9 | 0.73 | <0.05 | 0.11 | 0.23 | 0.18 |

Stability at 30° C./65% RH

| | | | | | | | | | |
|---|---|---|---|---|---|---|---|---|---|
| 0 | 36/60/64/68/73 | 392.0 | 61.0 | 19.8 | 0.66 | <0.05 | 0.17 | 0.07 | 0.24 |
| 2 | 37/59/65/67/70 | 393.0 | 64.0 | 19.9 | 0.49 | <0.05 | 0.10 | | 0.20 |
| 3 | 49/77/82/86/89 | 392.0 | 55.0 | 20.2 | 0.89 | | 0.13 | 0.30 | 0.23 |

Stability at 40° C./75% RH

| | | | | | | | | | |
|---|---|---|---|---|---|---|---|---|---|
| 0 | 36/60/64/68/73 | 392.0 | 61.0 | 19.8 | 0.66 | <0.05 | 0.17 | 0.07 | 0.24 |
| 1 | 37/65/72/73/75 | 393.0 | 60.0 | 19.5 | 0.55 | <0.05 | 0.10 | 0.09 | 0.19 |
| 2 | 40/62/68/69/72 | 392.0 | 59.0 | 19.7 | 0.54 | <0.05 | 0.14 | ??? | 0.21 |
| 3 | 52/80/88/90/92 | 388.0 | 66.0 | 19.9 | 1.10 | | 0.21 | 0.44 | 0.23 |
| Lipitor | 65/82/84/85/84 | | | | | | | | |

The results of Experiment 5 showed the following. The production of the cores of preferred embodiments of the formulation according to the present invention was easily scaled-up to pilot plant scale. The granulation process was particularly easily performed both in high shear and low shear granulation equipment. The properties of the cores of these formulations when produced at a pilot plant scale, especially their dissolution profile and disintegration time, were equivalent to those of Lipitor® independently from the pH of the dissolution test medium. The properties of these cores were maintained regardless of the kind of granulation equipment used (low shear or high shear granulator) and from the kind of Atorvastatin Calcium used (amorphous or crystalline).

The stability of the cores when suitably packaged in Alu/Alu blisters (as Lipitor®) was very good for all properties tested (even for impurities) when crystalline form VI Atorvastatin Calcium was used, even when the LOD of the cores was as high as 5% at T=0. When amorphous Atorvastatin Calcium was used, the stability of the cores was very good for all properties tested, although improvement may optionally and preferably be achieved for reducing impurities by decreasing the LOD of the cores at t=0 and evaluating the optimum Starch/Lactose ratio to be used in the formulation. Alternatively or additionally, stability may be improved by encapsulating the formulation in a gelatin capsule, as opposed to tablet compression, to decrease the influence of the excipients on the active ingredient.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-142. (canceled)

143. A stable pharmaceutical formulation comprising a pharmaceutically acceptable form of atorvastatin as active ingredient, and at least one major excipient selected from the group consisting of starch, pregelatinized starch or lactose or a combination thereof.

144. The formulation of claim 143, wherein the major excipient is present in an amount of at least about 30% to 90%.

145. The formulation of claim 144, wherein the major excipient comprises a plurality of major excipients, and the amount represents a total amount of the plurality of major excipients combined.

146. The formulation of claim 145, wherein the major excipient comprises a combination of the lactose and the pregelatinized starch in a ratio of from about 95/5 to about 5/95 weight percent of the formulation.

147. The formulation of claim 143, wherein the form of atorvastatin is determined according to one or more of a salt, a crystalline form or an amorphous form, alone or in combination.

148. The formulation of claim 147, wherein atorvastatin comprises an atorvastatin alkaline earth metal salt.

149. The formulation of claim 148, wherein the atorvastatin comprises crystalline atorvastatin calcium form VI or amorphous atorvastin as an active ingredient.

150. The formulation of claim 149, wherein the atorvastatin is present in an amount of from about 1% to about 50% weight per weight according to the weight of the base.

151. The formulation of claim 143, further comprising one or more of HPC, HPMC, PVP, crospovidone, a nonionic surfactant, magnesium stearate or silica as a minor excipient in an amount of up to about 35%.

152. The formulation of claim 143, further comprising one or more of a lubricant, a disintegrant, a filler, a binder, a gel forming ingredient, a tabletting aid, a glidant or a surfactant as a minor excipient wherein the total amount of minor excipients is up to 50%.

153. A pharmaceutical formulation of atorvastatin or any acceptable salt thereof which formulation is free of any stabilizer.

154. The formulation of claim 153, further comprising one or more of a lubricant, a disintegrant, a filler, a binder, a gel forming ingredient, a tabletting aid, a glidant or a surfactant.

155. The formulation of claim 154, further comprising a gel forming agent selected from the group consisting of a cellulose derivative, a vinyl polymer, an acrylic polymer or copolymer, a gum, a protein, a polysaccharide, a polyaminoacid, a polyalcohols and a polyglycol.

156. The formulation of claim 155, wherein the vinyl polymer is selected from the group consisting of polyvinylpyrrolidone and polyvinyl alcohol, the cellulose derivative is selected from the group consisting of methylcellulose, carboxymethylcellulose, hydroxypropylcellulose, hydroxypropylmethylcellulose, and hydroxyethylcellulose, the acrylic polymer or copolymer is selected from the group consisting of an acrylic acid polymer, carbopol, a methacrylic acid copolymer, and ethyl acrylate-methyl methacrylate copolymer, the gum is selected from the group consisting of guar gum, arabic gum, and xanthan gum, the protein is selected from the group consisting of gelatin and collagen, or the polysaccharide is selected from the group consisting of pectin, pectic acid, alginic acid, and sodium alginate.

157. A method for producing a stable pharmaceutical formulation comprising atorvastatin or salts thereof as active ingredient, the method comprising wet granulating atorvastatin with the proviso that the formulation is essentially free of croscarmellose or microcrystalline cellulose or any mono and/or di and/or tri valent metals containing excipients during the wet steps of the production process.

158. The method of claim 157, wherein the formulation is essentially free of CaCO₃ or other stabilizers.

159. The method of claim 157, wherein the formulation further comprises at least one major excipient in an amount of at least about 30%, wherein the at least one major excipient is granulated with the atorvastatin.

160. The method of claim 159, wherein the at least one major excipient comprises one or more of starch, pregelatinized starch or lactose.

161. A method for producing a stable pharmaceutical formulation according to claim 143, the method comprising granulating atorvastatin with at least one major excipient comprising one or more of starch, pregelatinized starch or lactose.

162. The method of claim 161, wherein the granulating comprises wet granulating.

163. A method for producing a stable pharmaceutical formulation comprising atorvastatin or salts thereof as active ingredient, the method comprising:
a) wet granulating atorvastatin with at least one excipient, wherein the at least one excipient is free of an incompatible excipient to form a granulate; and
b) after the wet granulation, adding an incompatible excipient to the granulate.

164. The method of claim 163, wherein the incompatible excipient is selected from the group consisting of croscarmellose sodium, carmellose calcium, or sodium starch glycolate.

165. The method of claim 164, wherein the minor incompatible excipient is present in an amount of up to about 10%.

166. The method of claim 165, wherein an amount of the minor incompatible excipient is determined according to a form of the atorvastatin.

167. A stable formulation according to claim 143, comprising crystalline atorvastatin calcium form VI with one or more of lactose, starch and pregelatinized starch, free of croscarmellose sodium, carmellose calcium, sodium starch glycolate or stearic acid.

168. The formulation of claim 167, further comprising a binder selected from the group consisting of HPC, HPMC and PVP; crospovidone, a non-ionic surfactant, magnesium stearate; silica, microcrystalline cellulose and mannitol.

169. A stable formulation according to claim 143, comprising amorphous atorvastatin calcium with one or more of lactose, starch and pregelatinized starch, free of croscarmellose sodium, carmellose calcium, sodium starch glycolate or stearic acid.

170. The formulation of claim 169, further comprising a binder selected from the group consisting of HPC, HPMC and PVP; crospovidone, a non-ionic surfactant, magnesium stearate, silica, microcrystalline cellulose and mannitol.

171. A method for producing a stable pharmaceutical formulation according to claim 153, the method comprising wet granulating atorvastatin with the proviso that the formulation is essentially free of a stabilizer.

172. The method of claim 172 wherein the formulation is essentially free of CaCO₃ but includes a disintegrant in an amount of up to about 15%.

173. The formulation of claim 172, wherein the disintegrant comprises crospovidone.

174. The formulation of claim 143, wherein the formulation is uncoated.

175. The formulation according to claim 143, further comprising an enteric coating, a film coating or a coating for providing one of modified release, delayed release, controlled release, slow release, sustained release, extended release, delayed controlled or sustained release, or extended release, delayed burst release, delayed fast or rapid release of atorvastatin.

176. The formulation of claim 175, wherein the major excipient and the atorvastatin are located in a core, and wherein the core further comprises at least one release controlling agent selected from the group consisting of methylcellulose, carboxymethylcellulose, hydroxypropylcellulose, hydroxypropylmethylcellulose, hydroxyethylcellulose; vinyl polymers; acrylic polymers and copolymers; natural and synthetic gums; gelatin, collagen, proteins, polysaccharides; and mixtures thereof.

177. The formulation of claim 172 which provides a lower dose of atorvastatin or salt thereof relative to conventional immediate release formulations, and releases atorvastatin in the lower gastrointestinal tract, in the small intestine, or in the colon of a subject.
